(19) United States
(12) Patent Application Publication
(10) Pub. No.: US 2022/0117964 A1
(43) Pub. Date: Apr. 21, 2022

(54) COMBINATION OF FAK INHIBITOR AND BTK INHIBITOR FOR TREATING A DISEASE

(71) Applicants: ASCENTAGE PHARMA (SUZHU) CO., LTD., JIANGSU (CN); ASCENTAGE PHARMA GROUP CORP LIMITED, HONG KONG (CN)

(72) Inventors: DAJUN YANG, JIANGSU (CN); MIAOZHEN QIU, JIANGSU (CN); QIUYUN LUO, JIANGSU (CN); SUNA ZHOU, JIANGSU (CN); LIN ZHANG, JIANGSU (CN); XIANGLEI YAN, JIANGSU (CN); LUPING YUAN, JIANGSU (CN); YUXIN ZHANG, JIANGSU (CN)

(21) Appl. No.: 17/413,319

(22) PCT Filed: Jun. 24, 2020
(86) PCT No.: PCT/CN2020/097992

§ 371 Date: Jun. 11, 2021

(30) Foreign Application Priority Data
Jun. 25, 2019 (CN) PCT/CN2019/092795

(51) Int. Cl. A61K 31/506; A61P 35/00

(52) U.S. Cl. CPC A61K 31/506; A61P 35/00

(57) ABSTRACT

A combination comprising a FAK inhibitor and a BTK inhibitor, a pharmaceutical composition and a kit, and a method for treating a disease such as esophageal cancer using the combination.

### TECHNICAL FIELD

The present invention relates to a combination comprising a FAK inhibitor and a BTK inhibitor, and the use of the combination to treat a disease.

### BACKGROUND ART

Esophageal cancer is a common tumor of the digestive tract, and about 300,000 people worldwide die from esophageal cancer every year. Esophageal squamous cell carcinoma (ESCC) is a fatal disease with poor prognosis and lacks effective targeting therapies. FAK overexpression is closely related to esophageal cancer cell differentiation, tumor invasion and metastasis. Approximately 60% of patients with esophageal cancer have high FAK expression and their 5-year survival rate is only half that of non-FAK high expression patients (38% vs. 69%). FAK inhibitors can reduce tumor cell proliferation and accelerate apoptosis, but it can only delay tumor growth in esophageal cancer tumor models with limited effects.

Bruton's tyrosine kinase (BTK) belongs to the Tec family. It consists of a unique N-terminal domain, namely PH (pleckstrin homology) domain, TH (Tec homology) homology region, SH3 (Src homology 3) domain, SH2 (Src homology 2) domain, and catalytic domain, which is called SH 1/TK (Src homology 1/Tyrosine kinase) domain or kinase domain composition (Akinleye et al: Ibrutinib and novel BTK inhibitors in clinical development, Journal of Hematology & Oncology 2013, 6:59). During the normal development of B lymphocytes, the correct expression of different protein regions of BTK gene plays a key role in B cell function and multiple transduction pathways.

The evidences for the role of BTK in autoimmune diseases have been provided by the experiments of BTK-deficient mouse and BTK-adequate mouse model (Kil L P, et al: Bruton's tyrosine kinase mediated signaling enhances leukemogenesis in a mouse model for chronic lymphocytic leukemia. Am J Blood Res 2013, 3(1): 71-83.). In a chronic lymphocytic leukemia (CLL) mouse model, the chronic lymphocytic leukemia was completely abolished in BTK-deficient mice, while the BTK overexpression accelerated leukemia and increased mortality.

Recent research of the research team led by Professor Christopher Lord of the London Cancer Institute in the United Kingdom shows that BTK is expected to become a new target for the treatment of esophageal cancer. The BTK inhibitor ibrutinib has been performed in clinical trials of cancer patients withMYC and ERBB2 amplification, but its action mechanism is unknown. Professor Lord has started a phase II clinical trial to further verify the effect of ibrutinib on esophageal cancer cells with upregulated MYC and ERBB2 gene activity, but has not yet established the effect of Ibrutinib on esophageal squamous cell carcinoma.

### SUMMARY OF THE INVENTION

According to one aspect of the invention, it provides a combination comprising a FAK inhibitor and a BTK inhibitor, which is used in the treatment of a cancer, a chronic autoimmune disorder, an inflammatory disorder, or a proliferative disorder.

According to one aspect of the invention, it provides a combination comprising a FAK inhibitor and a BTK inhibitor, which is used in the treatment of anaplastic large cell lymphoma, non-small cell lung cancer, diffuse large B cell lymphoma, inflammatory myofibroblastoma, anaplastic thyroid cancer, rhabdomyosarcoma, breast cancer, colorectal cancer, esophageal cancer (esophagus cancer), renal cell carcinoma, mantle cell lymphoma, chronic lymphocytic leukemia/small lymphocytic leukemia, chronic lymphocytic leukemia/small lymphocytic leukemia with 17p deletion, macroglobulinemia, borderline lymphoma, chronic graft-versus-host disease. FAK high-expression solid tumor, systemic lupus erythematosus (SLE) and rheumatoid arthritis (RA).

According to one aspect of the invention, it provides a combination comprising a FAK inhibitor and a BTK inhibitor, which is used in the treatment of esophageal cancer, systemic lupus erythematosus (SLE), and rheumatoid arthritis (RA). According to one aspect of the present invention, it provides a combination comprising a FAK inhibitor and a BTK inhibitor, which is used in the treatment of esophageal squamous cell carcinoma (ESCC).

According to the present invention, the FAK inhibitor includes a compound of Formula I, Formula II, Formula III, Formula IV, Formula V, Formula VI, or a pharmaceutically acceptable salt or solvate thereof, for example, 5-chloro-N²-(2-isopropoxy-5-methyl-4-(1-(tetrahydro-2H-pyran-4-yl)-1,2,3,6-tetrahydropyridin-4-yl)phenyl)-N⁴-(2-(isopropylsulfonyl) phenyl)pyrimidine-2,4-diamine (Compound 5) and pharmaceutically acceptable salts and solvates thereof.

The structure of Compound 5 is as follows:

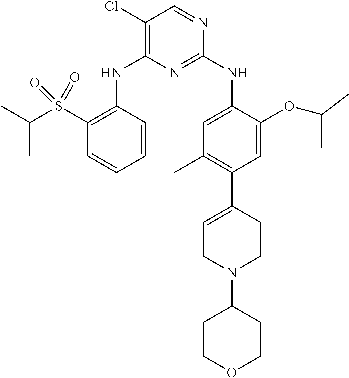 [SMILES: Cc1cc(Nc2ncc(Cl)c(Nc3ccccc3S(=O)(=O)C(C)C)n2)c(OC(C)C)cc1C1=CCN(C2CCOCC2)CC1]

According to the present invention, the BTK inhibitor includes: Ibrutinib, ICP-022, Acalabrutinib (ACP-196), BGB3111, ONO/GS-4059, Spebrutinib (CC-292 or AVL-292), CNX-774, Olmutinib (HM61713, BI1482694), M7583, HM71224, PCI-32765 Racemate, GDC-0853, ONO-4059, Zanubrutinib, RN486, PCI-32765, CGI-1746, QL47, LFM-A13, (±)-Zanubrutinib, SNS-062, BMS-935177, Btk inhibitor 2, Evobrutinib, Ibrutinib-biotin, BMX-IN-1, GDC-0834, and CB1763, wherein Ibrutinib and Ibrutinib-biotin are preferred. According to the present invention, a BTK inhibitor such as Ibrutinib not only has an antitumor effect in MYC/ERBB2 amplification or high-expression esophageal cancer, but also has a significant inhibitory effect on tumor cells in ESCC with EGFR expression. According to the present invention, a BTK inhibitor such as Ibrutinib can significantly reduce the protein expression of phosphorylated-EGFR, as well as the downstream protein expression of phosphorylated-AKT.

The combination of a BTK inhibitor such as Ibrutinib and a FAK inhibitor such as Compound 5 can achieve a more significant effect of reducing the expression of phosphorylated-AKT protein. Therefore, according to the present invention, a combination of a BTK inhibitor such as Ibrutinib and a FAK inhibitor such as Compound 5 is provided, which may become a new therapy for EGFR-expressing ESCC.

In some embodiments, the combination comprising a FAK inhibitor and a BTK inhibitor is in the form of a pharmaceutical composition.

In some embodiments, the FAK inhibitor and the BTK inhibitor are each presented as separate formulations in a kit.

In some embodiments, the FAK inhibitor and the BTK inhibitor are administrated simultaneously or sequentially.

In some embodiments, the composition according to the present invention comprises a pharmaceutically acceptable carrier, diluent or excipient.

In some embodiments, the composition according to the present invention is in the form of a tablet, a capsule, a granule, a syrup, a powder, a lozenge, a sachet, a cachet, an elixir, a suspension, an emulsion, a solution, a syrup, an aerosol, an ointment, a cream and an injection.

A second aspect according to the present invention provides a use of a composition or a kit comprising a FAK inhibitor and a BTK inhibitor in the manufacture of a medicament for treating a disease, including an esophageal cancer (e.g., esophageal squamous cell carcinoma (ESCC)).

A third aspect according to the present invention provides a method for treating a disease, including an esophageal cancer (e.g., esophageal squamous cell carcinoma (ESCC)), comprising administering to a subject in need thereof a therapeutically effective amount of a FAK inhibitor and a BTK Inhibitor.

### BRIEF DESCRIPTION OF THE DRAWINGS

FIG. 1A shows the expression difference of FAK gene between normal esophageal tissue and esophageal cancer tissue by analyzing the TCGA database source information in the GEPIA database (http://gepia.cancer-pku.cn/).

FIG. 1B shows the correlation between the FAK gene and the EGFR gene in esophageal cancer determined by analyzing the TCGA database source information in the GEPIA database (http://gepia.cancer-pku.cn/) using Pearson test.

FIG. 1C shows the proteins extracted from 6 esophageal cancer cell lines (TE-10, TE-1, YES-2, KYSE-520, KYSE-510, KYSE-150) in the logarithmic proliferation phase within 4 passages of fresh resuscitation, and the basic protein expression levels detected by Western blot method for the following proteins: EGFR, phosphorylated-EGFR (p-EGFR(Tyr1068)), FAK, phosphorylated-FAK (p-FAK(Tyr397)), BTK, C-Myc, phosphorylated-C-Myc (p-C-Myc), in which f3-tubulin was used as internal reference protein.

FIG. 1D shows the cytotoxic effects of Compound 5 at different concentrations on different esophageal cancer cell lines shown in FIG. 1C, which indicate the difference in activity of Compound 5 on different esophageal cancer cells.

FIG. 1E shows the cytotoxic effect of Ibrutinib at different concentrations on different esophageal cancer cell lines shown in FIG. 1C, which indicate the difference in activity of Compound 5 on different esophageal cancer cells.

FIG. 2A shows the cell growth inhibition effects of Compound 5 and Ibrutinib alone and in combination on ESCC cell line KYSE-150.

FIG. 2B shows the cell growth inhibitory effects of Compound 5 and Ibrutinib alone and in combination on ESCC cell line YSE-2.

FIG. 2C shows the cell growth inhibitory effects of Compound 5 and Ibrutinib alone and in combination on ESCC cell line KYSE-520.

FIG. 2D shows that the combination of Compound 5 and Ibrutinib exhibits a significant synergistic effect on cell cycle arrest.

FIG. 2E shows that the combination of Compound 5 and Ibrutinib exhibits a significant synergistic effect on apoptosis induction.

FIG. 3A shows the inhibitory effect of Compound 5 combined with Ibrutinib on esophageal cancer cell proliferation detected by clone formation assay.

FIG. 3B shows the inhibitory effect of Compound 5 combined with Ibrutinib on esophageal cancer cell migration detected by cell migration (Transwell) assay.

FIGS. 3C and 3D show the effect of Compound 5 combined with Ibrutinib on esophageal cancer cell-associated pathway proteins by Western blot assay.

FIG. 4A shows the effect of Compound 5 and Ibrutinib administrated alone and in combination on tumor growth in vivo, indicating that the administration in combination significantly inhibits the tumor growth in vivo.

FIG. 4B shows the effect of Compound 5 and Ibrutinib alone and in combination on body weight in mice.

### DEFINITION

Unless defined otherwise below, all technical and scientific terms used herein are intended to have the same meaning as commonly understood by those skilled in the art. References to the techniques used herein are intended to refer to techniques that are generally understood in the art, including those alternative or equivalent techniques that are obvious to those skilled in the art. Although it is believed that the following terms can be well understood by those skilled in the art, the following definitions are set forth to better explain the present invention.

As used herein, the terms “comprising,” “including,” “having,” “containing,” or “referring to” and other variations thereof used herein are inclusive or open-ended and do not exclude other elements or method steps that are not listed.

As used herein, “FAK” refers to focal adhesion Kinase, and “FAK inhibitor” refers to an agent that has an inhibitory effect on FAK. In some embodiments, the FAK inhibitor also has an inhibitory effect on one or more of other targets (e.g., ALK and/or ROS1).

The term “BTK inhibitor” as used herein refers to a substance that inhibits the activity of a BTK enzyme, or a substance that degrades a BTK enzyme, or a genetic tool that reduces the level of a BTK enzyme.

The term “pharmaceutically acceptable salt” as used herein refers to a salt of a free acid or a free base, which is typically prepared by reacting a free base with a suitable organic or inorganic acid or by reacting an acid with a suitable organic or inorganic base. The term can be used for any compound in the present invention. Representative salts include: acetate, benzenesulfonate, benzoate, bicarbonate, bisulfate, hydrogen tartrate, borate, bromide, calcium edetate, camphorsulfonate, carbonate, chloride, clavulanate, citrate, dihydrochloride, edetate, ethanedisulfonate, estolate, esylate, fumarate, glucoheptanoate, gluconate, glutamate, glycol lylarsanilate, hexylresorcinate, hydrabamine, hydrobromide, hydrochloride, pamoate, iodide, isethionate, lactate, lactobionate, laurate, malate, maleate, mandelate, methanesulfonate, methylbromate, methylnitrate, methosulfate, monopotassium maleate, mucate, naphthalenesulfonate, nitrate, N-methylglucamine salt, oxalate, pamoate, palmitate, pantothenate, phosphate/bisphosphate, polygalacturonate, potassium salt, salicylate, sodium salt, stearate, subacetate, succinate, tannate, tartrate, teoclate, p-toluenesulfonate, triethiodide, trimethylamine salt, and valerate. When an acidic substituent such as —COOH is present, an ammonium salt, a morpholine salt, a sodium salt, a potassium salt, a barium salt, a calcium salt, and the like can be formed for use in a dosage form. When a basic group (for example, that in a limonin compound or 1,1-dimethyl biguanide), such as an amino group or a basic heteroaryl group such as pyridyl, is present, an acidic salt such as the hydrochloride, hydrobromate, phosphate, sulfate, trifluoroacetate, trichloroacetate, acetate, oxalate, maleate, pyruvate, malonate, succinate, citrate, tartrate, fumarate, mandelate, benzoate, cinnamate, mesylate, ethanesulfonate, picrate, etc.

The term “treating” as used herein refers to reducing, alleviating or ameliorating a symptom of a disease or disorder, improving a symptom caused by underlying metabolism, inhibiting a disease or symptom, such as preventing a development of a disease or disorder, alleviating a disease or disorder, causing a regression of a disease or disorder, alleviating a condition caused by a disease or disorder, or preventing a symptom of a disease or disorder.

The term “solvate” as used herein is a combination, physical conjugate, and/or solvate of a compound and a solvent molecule involved in the present invention, such as a disolvate, a monosolvate, a semisolvate. The compound involved in the present invention can be in a solvated form with a pharmaceutically acceptable solvent such as water, methanol, ethanol, etc., which does not significantly affect the pharmacological activity or toxicity of the compound and thus can function as a pharmacological equivalent.

The term “subject” as used herein is meant to include humans (e.g., patients) and animals (e.g., mice, rats, dogs, cats, rabbits, chickens or monkeys, etc.). When the subject is a human patient (which body weight is usually calculated as 60 kg), unless otherwise stated, the dose according to the present invention may be obtained by conversion with a conversion factor of experimental animal (for example, human dose=mouse dose/12.3) (refer to Kin Tam. “Estimating the “First in human” dose—a revisit with particular emphasis on oncology drugs, ADMET & DMPK 1(4)(2013) 63-75). According to general common sense, the dosage is reasonably adjusted by those skilled in the art according to the specific weight of the subject, the kind and severity of the disease, and other factors, and these adjusted technical solutions fall within the scope of the technical solutions claimed in the present invention.

The term “effective amount” or “therapeutically effective amount” as used herein refers to a sufficient amount (e.g., a dose) of a medicament or compound as administrated that alleviates to some extent one or more symptoms of the disease or disorder to be treated. The result may be a reduction and/or amelioration in a condition or cause of a disease or any other desired alteration of biological system. For example, an “effective amount” for therapeutic use is an amount of a compound or medicament (e.g., a pharmaceutical composition as claimed in the present application) that is provided to significantly reduce a clinical symptom of a disease or disorder without causing an excessive toxic or side effect.

The term “dose” as used herein refers to a weight of the active substance (e.g., milligrams (mg)) per kilogram (kg) of the subject's body weight.

The term “IC₅₀” as used herein refers to an amount, concentration, or dose of a particular test compound or medicament that achieves an inhibition of 50% of the maximum effect in an experiment measuring such effect, such as the inhibition of FAK or BTK.

The term “room temperature” as used herein refers to 25° C.±1° C. At the same time, if the experimental temperature is not specified, it is all room temperature.

The term “about” as used herein refers to ±10%, more preferably ±5%, and most preferably ±2%, of the value defined by the term, so that those skilled in the art can clearly determine the range of the term “about” according to the defined value.

The term “ibrutinib” as used herein is a compound having the following structure

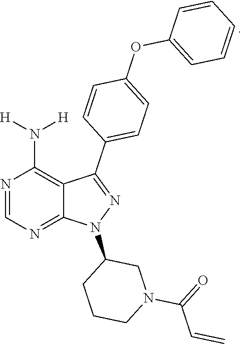 [SMILES: C=CC(=O)N1CCC[C@@H](n2nc(-c3ccc(Oc4ccccc4)cc3)c3c(N)ncnc32)C1]

### Pharmaceutical Compositions and Kits

A first aspect of the present invention provides a pharmaceutical composition, comprising a FAK inhibitor and a BTK inhibitor.

In some embodiments, the FAK inhibitor is a compound of Formula I or a pharmaceutically acceptable salt or solvate thereof:

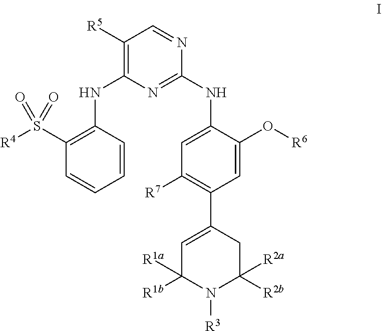

wherein:

R^{1a} and R^{1b} are independently selected from the group consisting of hydrogen, C₁₋₆ alkyl and C₃₋₈ cycloalkyl;

R^{2a} and R^{2b} are independently selected from the group consisting of hydrogen, C₁₋₆ alkyl and C₃₋₈ cycloalkyl;

R³ is selected from the group consisting of hydrogen, C₁₋₆ alkyl, C₃₋₆ cycloalkyl, and 4- to 8-membered heterocyclyl;

R⁴ is selected from the group consisting of C₁₋₄ alkyl and C₃₋₆ cycloalkyl;

R⁵ is halogen;

R⁶ is selected from the group consisting of C₁₋₄ alkyl and C₃₋₆ cycloalkyl; and

R⁷ is selected from the group consisting of hydrogen, C₁₋₄ alkyl and C₃₋₆ cycloalkyl; with proviso that when R^{1a}, R^{1b}, R^{2a} and R^{2b} are each hydrogen, then R³ is selected from the group consisting of C₃₋₆ cycloalkyl and 4- to 8-membered heterocyclyl.

In some embodiments, the FAK inhibitor is a compound of Formula II or a pharmaceutically acceptable salt or solvate thereof:

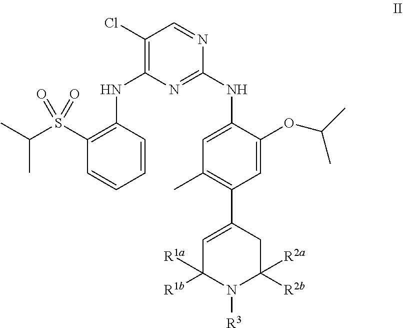

wherein:

R^{1a} and R^{1b} are independently selected from the group consisting of hydrogen, C₁₋₄ alkyl and C₃₋₆ cycloalkyl;

R^{2a} and R^{2b} are independently selected from the group consisting of hydrogen, C₁₋₄ alkyl and C₃₋₆ cycloalkyl; and

R³ is selected from the group consisting of hydrogen, C₁₋₄ alkyl, C₃₋₆ cycloalkyl, and 4- to 8-membered heterocyclyl.

In some embodiments, the FAK inhibitor is a compound of Formula III or a pharmaceutically acceptable salt or solvate thereof:

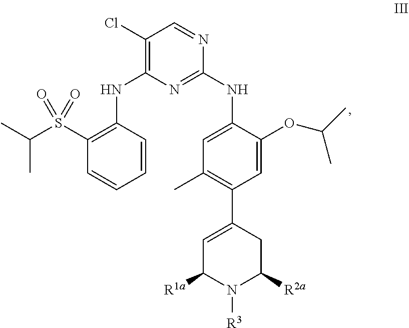

wherein:

R^{1a} and R^{2a} are each independently selected from the group consisting of C₁₋₄ alkyl and C₃₋₆ cycloalkyl; and

the compound has an enantiomeric excess of 90% or higher.

In some embodiments, the FAK inhibitor is a compound of Formula IV or a pharmaceutically acceptable salt or solvate thereof:

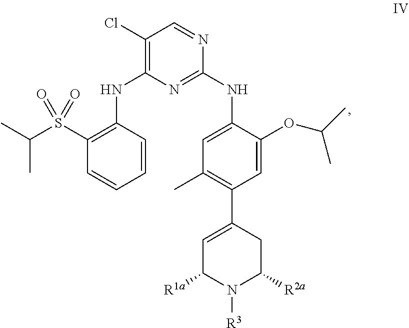

wherein:

R^{1a} and R^{2a} are each independently selected from the group consisting of C₁₋₄ alkyl and C₃₋₆ cycloalkyl; and

the compound has an enantiomeric excess of 90% or higher.

In some embodiments, the FAK inhibitor is a compound of Formula V or a pharmaceutically acceptable salt or solvate thereof:

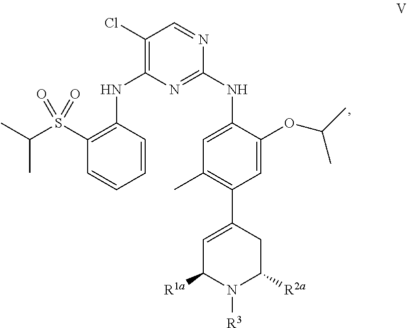

wherein:

R^{1a} and R^{2a} are each independently selected from the group consisting of C₁₋₄ alkyl and C₃₋₆ cycloalkyl; and

the compound has an enantiomeric excess of 90% or higher.

In some embodiments, the FAK inhibitor is a compound of Formula VI or a pharmaceutically acceptable salt or solvate thereof:

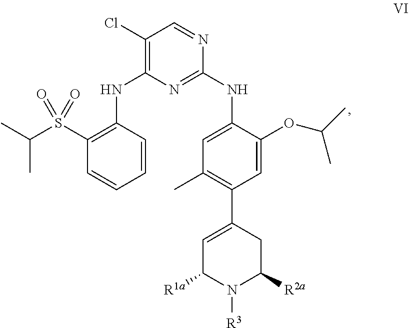

wherein:

R^{1a} and R^{2a} are each independently selected from the group consisting of C₁₋₄ alkyl and C₃₋₆ cycloalkyl; and

the compound has an enantiomeric excess of 90% or higher.

In some embodiments, the FAK inhibitor is a compound in the following table or a pharmaceutically acceptable salt or solvate thereof:

| No. | Structure | Name |
|---|---|---|
| 1 | 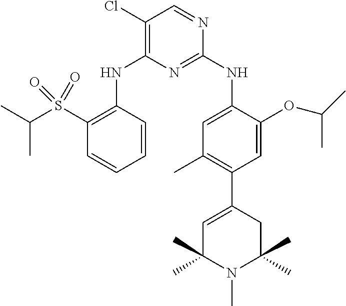 | 5-chloro-N²-(2-isopropoxy-5-methyl-4-(1,2,2,6,6- pentamethyl-1,2,3,6-tetrahydropyridin-4-yl)phenyl)-N⁴-(2- (isopropylsulfonyl)phenyl)pyrimidine-2,4-diamine; |
| 2 | 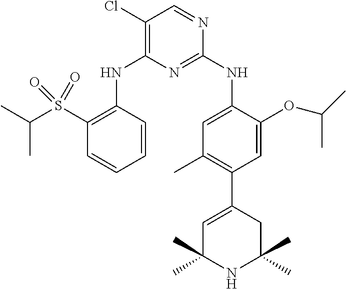 | 5-chloro-N²-(2-isopropoxy-5-methyl-4-(2,2,6,6-tetramethyl- 1,2,3,6-tetrahydropyridin-4-yl)phenyl)-N⁴-(2-(isopropyl- sulfonyl)phenyl)pyrimidine-2,4-diamine; |
| 3 | 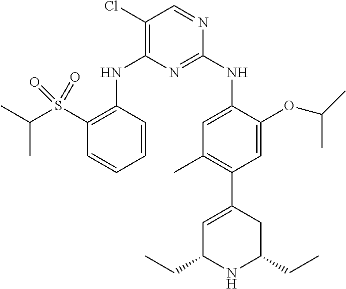 [SMILES: CC[C@H]1CC(c2cc(OC(C)C)c(Nc3ncc(Cl)c(Nc4ccccc4S(=O)(=O)C(C)C)n3)cc2C)=C[C@@H](CC)N1] | 5-chloro-N²-(4-((cis)-2,6-diethyl-1,2,3,6-tetrahydropyridin- 4-yl)-2-isopropoxy-5-methylphenyl)-N⁴-(2-(isopropyl- sulfonyl)phenyl)pyrimidine-2,4-diamine; |
| 4 | 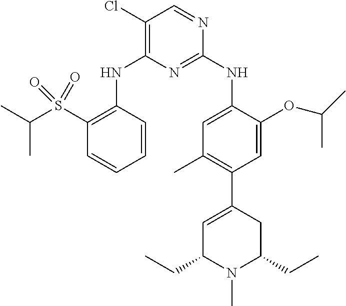 [SMILES: CC[C@H]1CC(c2cc(OC(C)C)c(Nc3ncc(Cl)c(Nc4ccccc4S(=O)(=O)C(C)C)n3)cc2C)=C[C@@H](CC)N1C] | 5-chloro-N²-(4-((cis)-2,6-diethyl-1-methyl-1,2,3,6- tetrahydropyridin-4-yl)-2-isopropoxy-5-methylphenyl)-N⁴- (2-(isopropylsulfonyl)phenyl)pyrimidine-2,4-diamine; |
| 5 | 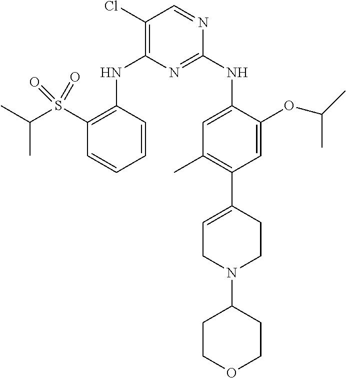 [SMILES: Cc1cc(Nc2ncc(Cl)c(Nc3ccccc3S(=O)(=O)C(C)C)n2)c(OC(C)C)cc1C1=CCN(C2CCOCC2)CC1] | 5-chloro-N²-(2-isopropoxy-5-methyl-4-(1-(tetrahydro-2H- pyran-4-yl)-1,2,3,6-tetrahydropyridin-4-yl)phenyl)-N⁴-(2- (isopropylsulfonyl)phenyl)pyrimidine-2,4-diamine; |
| 6 | 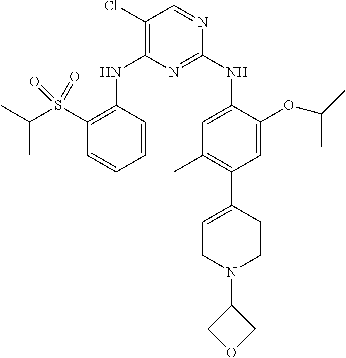 [SMILES: Cc1cc(Nc2ncc(Cl)c(Nc3ccccc3S(=O)(=O)C(C)C)n2)c(OC(C)C)cc1C1=CCN(C2COC2)CC1] | 5-chloro-N²-(2-isopropoxy-5-methyl-4-(1-(oxetan-3-yl)-1,2, 3,6-tetrahydropyridin-4-yl)phenyl)-N⁴-(2-(isopropyl- sulfonyl)phenyl)pyrimidine-2,4-diamine; |
| 7 | 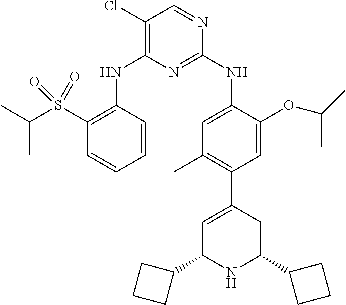 [SMILES: Cc1cc(Nc2ncc(Cl)c(Nc3ccccc3S(=O)(=O)C(C)C)n2)c(OC(C)C)cc1C1=C[C@@H](C2CCC2)N[C@@H](C2CCC2)C1] | 5-chloro-N²-(4-((cis)-2,6-bicyclobutyl-1,2,3,6- tetrahydropyridin-4-yl)-2-isopropoxy-5-methylphenyl)-N⁴- 2-(isopropylsulfonyl)phenyl)pyrimidine-2,4-diamine; |
| 8 | 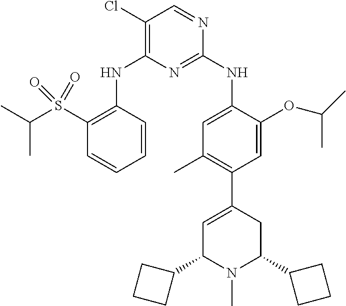 [SMILES: Cc1cc(Nc2ncc(Cl)c(Nc3ccccc3S(=O)(=O)C(C)C)n2)c(OC(C)C)cc1C1=C[C@@H](C2CCC2)N(C)[C@@H](C2CCC2)C1] | 5-chloro-N²-(4-((cis)-2,6-bicyclobutyl-1-methyl-1,2,3,6- tetrahydropyridin-4-yl)-2-isopropoxy-5-methylphenyl)-N⁴- (2-(isopropylsulfonyl)phenyl)pyrimidine-2,4-diamine; |
| 9 | 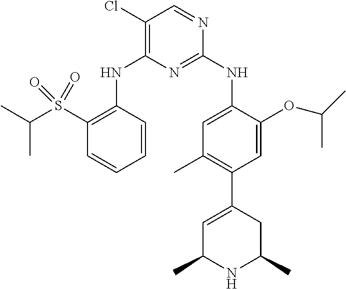 [SMILES: Cc1cc(Nc2ncc(Cl)c(Nc3ccccc3S(=O)(=O)C(C)C)n2)c(OC(C)C)cc1C1=C[C@H](C)N[C@H](C)C1] | 5-chloro-N²-(4-((cis)-2,6-dimethyl-1,2,3,6- tetrahydropyridin-4-yl)-2-isopropoxy-5-methylphenyl)-N⁴- 2-(isopropylsulfonyl)phenyl)pyrimidine-2,4-diamine; |
| 10 | 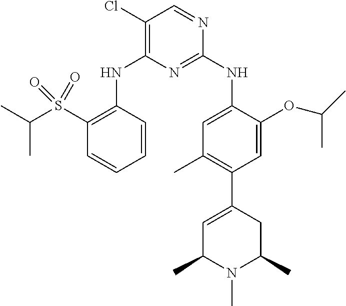 [SMILES: Cc1cc(Nc2ncc(Cl)c(Nc3ccccc3S(=O)(=O)C(C)C)n2)c(OC(C)C)cc1C1=C[C@H](C)N(C)[C@H](C)C1] | 5-chloro-N²-(2-isopropoxy-5-methyl-4-((cis)-1,2,6- trimethyl-1,2,3,6-tetrahydropyridin-4-yl)phenyl)-N⁴-(2- (isopropylsulfonyl)phenyl)pyrimidine-2,4-diamine; |
| 11 | 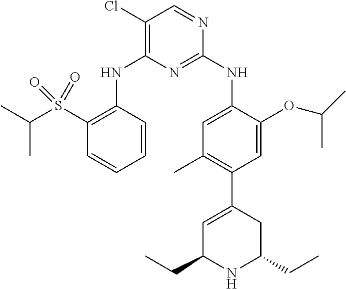 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1] | 5-chloro-N²-(4-((trans)-2,6-diethyl-1,2,3,6- tetrahydropyridin-4-yl)-2-isopropoxy-5-methylphenyl)-N⁴- 2-(isopropylsulfonyl)phenyl)pyrimidine-2,4-diamine; |
| 12 | 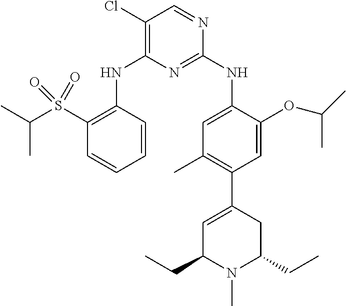 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1C] | 5-chloro-N²-(4-((trans)-2,6-diethyl-1-methyl-1,2,3,6- tetrahydropyridin-4-yl)-2-isopropoxy-5-methylphenyl)-N⁴- (2-(isopropylsulfonyl)phenyl)pyrimidine-2,4-diamine; |
| 13 | 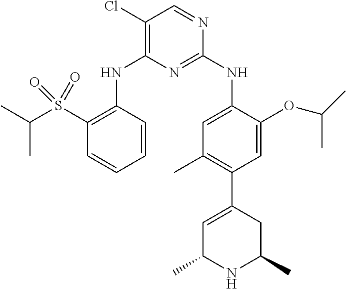 [SMILES: Cc1cc(Nc2ncc(Cl)c(Nc3ccccc3S(=O)(=O)C(C)C)n2)c(OC(C)C)cc1C1=C[C@@H](C)N[C@H](C)C1] | 5-chloro-N²-(4-((trans)-2,6-dimethyl-1,2,3,6- tetrahydropyridin-4-yl)-2-isopropoxy-5-methylphenyl)-N⁴- (2-(isopropylsulfonyl)phenyl)pyrimidine-2,4-diamine; |
| 14 | 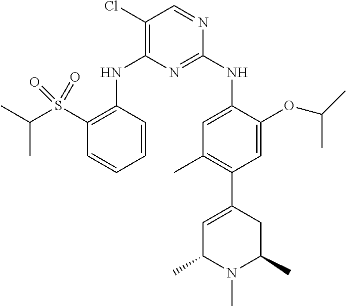 [SMILES: Cc1cc(Nc2ncc(Cl)c(Nc3ccccc3S(=O)(=O)C(C)C)n2)c(OC(C)C)cc1C1=C[C@@H](C)N(C)[C@H](C)C1] | 5-chloro-N²-(2-isopropoxy-5-methyl-4-((trans)-1,2,6- trimethyl-1,2,3,6-tetrahydropyridin-4-yl)phenyl)-N⁴-(2- (isopropylsulfonyl)phenyl)pyrimidine-2,4-diamine; |
| 15 | 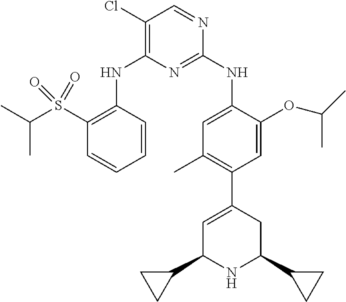 [SMILES: Cc1cc(Nc2ncc(Cl)c(Nc3ccccc3S(=O)(=O)C(C)C)n2)c(OC(C)C)cc1C1=C[C@H](C2CC2)N[C@H](C2CC2)C1] | 5-chloro-N²-(4-((cis)-2,6-bicyclopropyl-1,2,3,6- tetrahydropyridin-4-yl)-2-isopropoxy-5-methylphenyl)-N⁴- (2-(isopropylsulfonyl)phenyl)pyrimidine-2,4-diamine; |
| 16 | 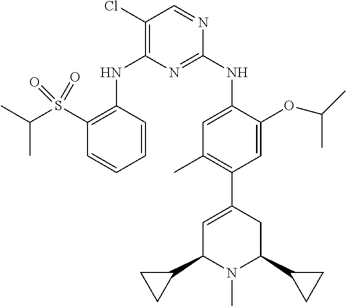 [SMILES: Cc1cc(Nc2ncc(Cl)c(Nc3ccccc3S(=O)(=O)C(C)C)n2)c(OC(C)C)cc1C1=C[C@H](C2CC2)N(C)[C@H](C2CC2)C1] | 5-chloro-N²-(4-((cis)-2,6-bicyclopropyl-1-methyl-1,2,3,6- tetrahydropyridin-4-yl)-2-isopropoxy-5-methylphenyl)-N⁴- (2-(isopropylsulfonyl)phenyl)pyrimidine-2,4-diamine; |
| 17 | 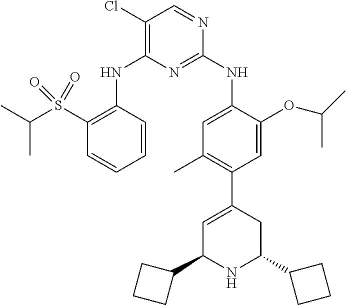 [SMILES: Cc1cc(Nc2ncc(Cl)c(Nc3ccccc3S(=O)(=O)C(C)C)n2)c(OC(C)C)cc1C1=C[C@H](C2CCC2)N[C@@H](C2CCC2)C1] | 5-chloro-N²-(4-((trans)-2,6-bicyclobutyl-1,2,3,6- tetrahydropyridin-4-yl)-2-isopropoxy-5-methylphenyl)-N⁴- (2-(isopropylsulfonyl)phenyl)pyrimidine-2,4-diamine; |
| 18 | 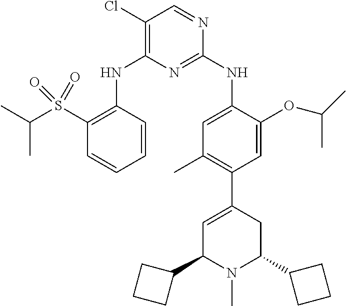 [SMILES: Cc1cc(Nc2ncc(Cl)c(Nc3ccccc3S(=O)(=O)C(C)C)n2)c(OC(C)C)cc1C1=C[C@H](C2CCC2)N(C)[C@@H](C2CCC2)C1] | 5-chloro-N²-(4-((trans)-2,6-bicyclobutyl-1-methyl-1,2,3,6- tetrahydropyridin-4-yl)-2-isopropoxy-5-methylphenyl)-N⁴- (2-(isopropylsulfonyl)phenyl)pyrimidine-2,4-diamine |
| 19 | 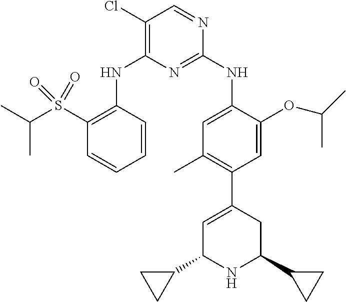 [SMILES: Cc1cc(Nc2ncc(Cl)c(Nc3ccccc3S(=O)(=O)C(C)C)n2)c(OC(C)C)cc1C1=C[C@@H](C2CC2)N[C@H](C2CC2)C1] | 5-chloro-N²-(4-((trans)-2,6-bicyclopropyl-1,2,3,6- tetrahydropyridin-4-yl)-2-isopropoxy-5-methylphenyl)-N⁴- (2-(isopropylsulfonyl)phenyl)pyrimidine-2,4-diamine; |
| 20 | 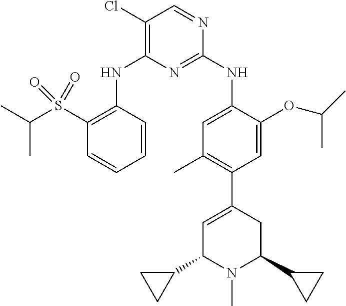 [SMILES: Cc1cc(Nc2ncc(Cl)c(Nc3ccccc3S(=O)(=O)C(C)C)n2)c(OC(C)C)cc1C1=C[C@@H](C2CC2)N(C)[C@H](C2CC2)C1] | 5-chloro-N²-(4-((trans)-2,6-bicyclopropyl-1-methyl-1,2,3,6- tetrahydropyridin-4-yl)-2-isopropoxy-5-methylphenyl)-N⁴- (2-(isopropylsulfonyl)phenyl)pyrimidine-2,4-diamine; |
| 21 | 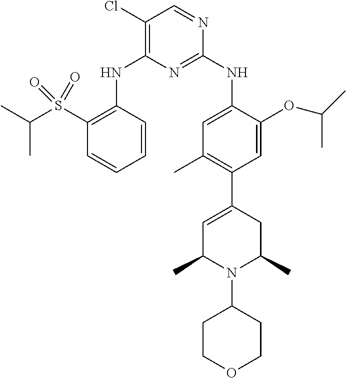 [SMILES: Cc1cc(Nc2ncc(Cl)c(Nc3ccccc3S(=O)(=O)C(C)C)n2)c(OC(C)C)cc1C1=C[C@H](C)N(C2CCOCC2)[C@H](C)C1] | 5-chloro-N²-(4-((cis)-2,6-dimethyl-1-(tetrahydro-2H- pyran-4-yl)-1,2,3,6-tetrahydropyridin-4-yl)-2-isopropoxy- 5-methylphenyl)-N⁴-(2-(isopropylsulfonyl)phenyl) pyrimidine-2,4-diamine; |
| 22 | 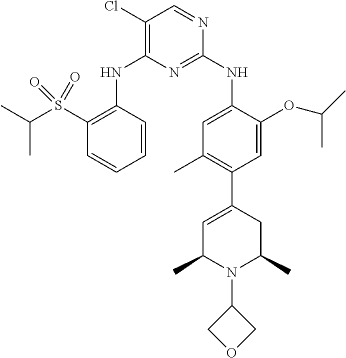 [SMILES: Cc1cc(Nc2ncc(Cl)c(Nc3ccccc3S(=O)(=O)C(C)C)n2)c(OC(C)C)cc1C1=C[C@H](C)N(C2COC2)[C@H](C)C1] | 5-chloro-N²-(4-((cis)-2,6-dimethyl-1-(oxetan-3-yl)-1,2,3,6- tetrahydropyridin-4-yl)-2-isopropoxy-5-methylphenyl)-N⁴- 2-(isopropylsulfonyl)phenyl)pyrimidine-2,4-diamine; |
| 23 | 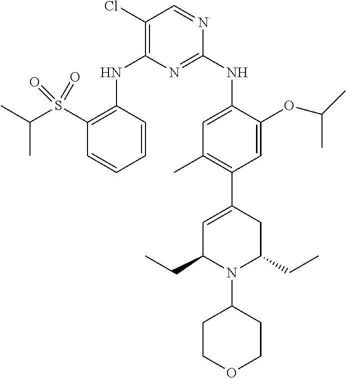 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1C1CCOCC1] | 5-chloro-N²-(4-((trans)-2,6-diethyl-1-(tetrahydro-2H- pyran-4-yl)-1,2,3,6-tetrahydropyridin-4-yl)-2-isopropoxy- 5-methylphenyl)-N⁴-(2-(isopropylsulfonyl)phenyl) pyrimidine-2,4-diamine; |
| 24 | 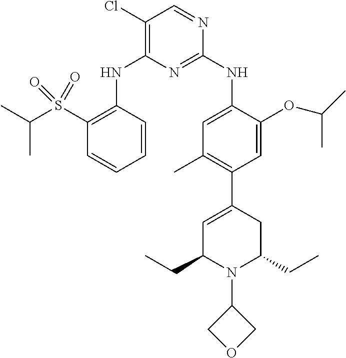 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1C1COC1] | 5-chloro-N²-(4-((2S,6S)-2,6-diethyl-1-(oxetan-3-yl)-1,2,3,6- tetrahydropyridin-4-yl)-2-isopropoxy-5-methylphenyl)-N⁴- (2-(isopropylsulfonyl)phenyl)pyrimidine-2,4-diamine; |
| 25 | 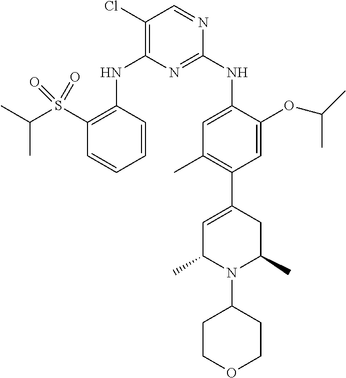 [SMILES: Cc1cc(Nc2ncc(Cl)c(Nc3ccccc3S(=O)(=O)C(C)C)n2)c(OC(C)C)cc1C1=C[C@@H](C)N(C2CCOCC2)[C@H](C)C1] | 5-chloro-N²-(4-((trans)-2,6-dimethyl-1-(tetrahydro-2H- pyran-4-yl)-1,2,3,6-tetrahydropyridin-4-yl)-2-isopropoxy- 5-methylphenyl)-N⁴-(2-(isopropylsulfonyl)phenyl) pyrimidine-2,4-diamine; |
| 26 | 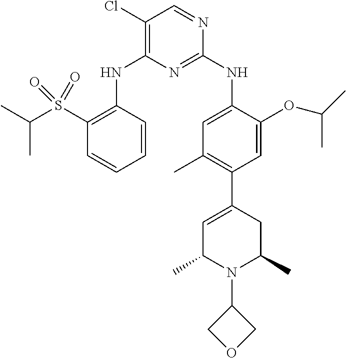 [SMILES: Cc1cc(Nc2ncc(Cl)c(Nc3ccccc3S(=O)(=O)C(C)C)n2)c(OC(C)C)cc1C1=C[C@@H](C)N(C2COC2)[C@H](C)C1] | 5-chloro-N²-(4-((trans)-2,6-dimethyl-1-(oxetan-3-yl)-1,2,3, 6-tetrahydropyridin-4-yl)-2-isopropoxy-5-methylphenyl)- N⁴-(2-(isopropylsulfonyl)phenyl)pyrimidine-2,4-diamine; |
| 27 | 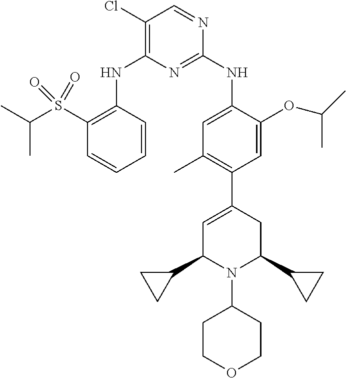 [SMILES: Cc1cc(Nc2ncc(Cl)c(Nc3ccccc3S(=O)(=O)C(C)C)n2)c(OC(C)C)cc1C1=C[C@H](C2CC2)N(C2CCOCC2)[C@H](C2CC2)C1] | 5-chloro-N²-(4-((cis)-2,6-bicyclopropyl-1-(tetrahydro-2H- pyran-4-yl)-1,2,3,6-tetrahydropyridin-4-yl)-2-isopropoxy- 5-methylphenyl)-N⁴-(2-(isopropylsulfonyl)phenyl) pyrimidine-2,4-diamine; |
| 28 | 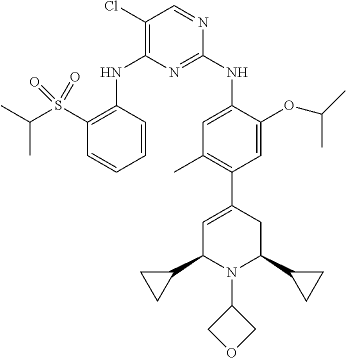 [SMILES: Cc1cc(Nc2ncc(Cl)c(Nc3ccccc3S(=O)(=O)C(C)C)n2)c(OC(C)C)cc1C1=C[C@H](C2CC2)N(C2COC2)[C@H](C2CC2)C1] | 5-chloro-N²-(4-((cis)-2,6-bicyclopropyl-1-(oxetan-3-yl)-1,2, 3,6-tetrahydropyridin-4-yl)-2-isopropoxy-5-methylphenyl)- N⁴-(2-(isopropylsulfonyl)phenyl)pyrimidine-2,4-diamine; |
| 29 | 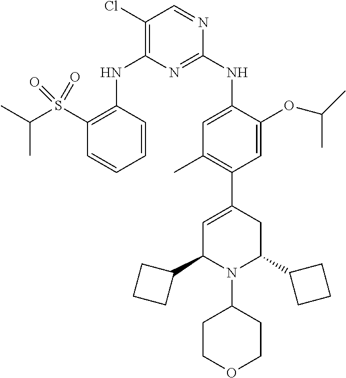 [SMILES: Cc1cc(Nc2ncc(Cl)c(Nc3ccccc3S(=O)(=O)C(C)C)n2)c(OC(C)C)cc1C1=C[C@H](C2CCC2)N(C2CCOCC2)[C@@H](C2CCC2)C1] | 5-chloro-N²-(4-((trans)-2,6-bicyclobutyl-1-(tetrahydro-2H- pyran-4-yl)-1,2,3,6-tetrahydropyridin-4-yl)-2-isopropoxy- 5-methylphenyl)-N⁴-(2-(isopropylsulfonyl)phenyl) pyrimidine-2,4-diamine; |
| 30 | 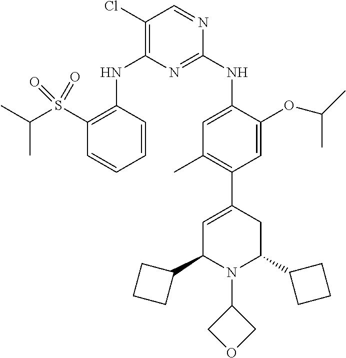 [SMILES: Cc1cc(Nc2ncc(Cl)c(Nc3ccccc3S(=O)(=O)C(C)C)n2)c(OC(C)C)cc1C1=C[C@H](C2CCC2)N(C2COC2)[C@@H](C2CCC2)C1] | 5-chloro-N²-(4-((trans)-2,6-bicyclobutyl-1-(oxetan-3-yl)-1, 2,3,6-tetrahydropyridin-4-yl)-2-isopropoxy-5- methylphenyl)-N⁴-(2-(isopropylsulfonyl)phenyl) pyrimidine-2,4-diamine; |
| 31 | 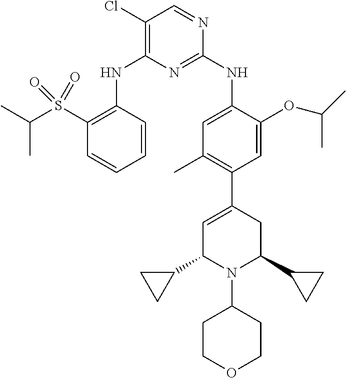 [SMILES: Cc1cc(Nc2ncc(Cl)c(Nc3ccccc3S(=O)(=O)C(C)C)n2)c(OC(C)C)cc1C1=C[C@@H](C2CC2)N(C2CCOCC2)[C@H](C2CC2)C1] | 5-chloro-N²-(4-((trans)-2,6-bicyclopropyl-1-(tetrahydro- 2H-pyran-4-yl)-1,2,3,6-tetrahydropyridin-4-yl)-2- isopropoxy-5-methylphenyl)-N⁴-(2-(isopropylsulfonyl) phenyl)pyrimidine-2,4-diamine; or |
| 32 | 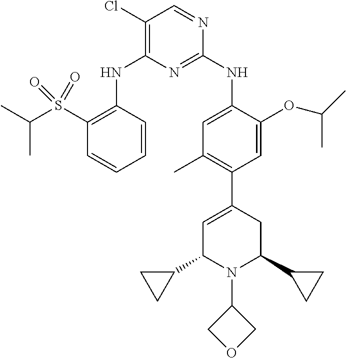 [SMILES: Cc1cc(Nc2ncc(Cl)c(Nc3ccccc3S(=O)(=O)C(C)C)n2)c(OC(C)C)cc1C1=C[C@@H](C2CC2)N(C2COC2)[C@H](C2CC2)C1] | 5-chloro-N²-(4-((trans)-2,6-bicyclopropyl-1-(oxetan-3-yl)- 1,2,3,6-tetrahydropyridin-4-yl)-2-isopropoxy-5- methylphenyl)-N⁴-(2-(isopropylsulfonyl)phenyl) pyrimidine-2,4-diamine. |

In some embodiments, the FAK inhibitor is 5-chloro-N²-(2-isopropoxy-5-methyl-4-(1-(tetrahydro-2H-pyran-4-yl)-1,2,3,6-tetrahydropyridin-4-yl)phenyl)-N⁴-(2-(isopropylsulfonyl) phenyl)pyrimidine-2,4-diamine (Compound 5) or a pharmaceutically acceptable salt or solvate thereof.

In some embodiments, the BTK inhibitor is selected from the group consisting of: Ibrutinib, ICP-022, Acalabrutinib (ACP-196), BGB3111, ONO/GS-4059, Spebrutinib (CC-292 or AVL-292), CNX-774, Olmutinib (HM61713, BI1482694), M7583, HM71224, PCI-32765 Racemate, GDC-0853, ONO-4059, Zanubrutinib, RN486, PCI-32765, CGI-1746, QL47, LFM-A13, (±)-Zanubrutinib, SNS-062, BMS-935177, Btk inhibitor 2, Evobrutinib, Ibrutinib-biotin, BMX-IN-1, GDC-0834 and CB1763.

In a preferred embodiment, the BTK inhibitor is preferably Ibrutinib or ICP-022.

In some embodiments, the pharmaceutical composition further comprises a pharmaceutically acceptable carrier, diluent or excipient.

In some embodiments, the pharmaceutical composition is in the form of a tablet, a capsule, a granule, a syrup, a powder, a lozenge, a sachet, a cachet, an elixir, a suspension, an emulsion, a solution, a syrup, an aerosol, an ointment, a cream and an injection.

The kit provided by the present invention comprises a FAK inhibitor and a BTK inhibitor, wherein the FAK inhibitor is preferably a FAK inhibitor as defined above, and optionally a pharmaceutically acceptable carrier, and the BTK inhibitor is preferably a BTK inhibitor as defined above, and optionally a pharmaceutically acceptable carrier.

In a preferred embodiment, the FAK inhibitor is 5-chloro-N²-(2-isopropoxy-5-methyl-4-(1-(tetrahydro-2H-pyran-4-yl)-1,2,3,6-tetrahydropyridin-4-yl)phenyl)-N⁴-(2-(isopropylsulfonyl) phenyl)pyrimidine-2,4-diamine (Compound 5) or a pharmaceutically acceptable salt or solvate thereof, and the BTK inhibitor is Ibrutinib.

In some embodiments, the FAK inhibitor and the BTK inhibitor are administrated simultaneously or sequentially.

### Treatment Methods and Uses

In another aspect, the present invention provides a method for treating a disease, comprising administering a therapeutically effective amount of a FAK inhibitor and/or a BTK inhibitor to a subject in need thereof, and the disease is selected from the group consisting of cancer, chronic autoimmune disease, inflammatory disease or proliferative disease.

In some embodiments, the FAK inhibitor is preferably a FAK inhibitor as defined above, and optionally a pharmaceutically acceptable carrier, and the BTK inhibitor is preferably a BTK inhibitor as defined above, and optionally a pharmaceutically acceptable carrier.

In some embodiments, the FAK inhibitor is 5-chloro-N²-(2-isopropoxy-5-methyl-4-(1-(tetrahydro-2H-pyran-4-yl)-1,2,3,6-tetrahydropyridin-4-yl)phenyl)-N⁴-(2-(isopropylsulfonyl) phenyl)pyrimidine-2,4-diamine (Compound 5) or a pharmaceutically acceptable salt or solvate thereof, and the BTK inhibitor is Ibrutinib.

In some embodiments, the disease is selected from the group consisting of anaplastic large cell lymphoma, non-small cell lung cancer, diffuse large B cell lymphoma, inflammatory myofibroblastoma, anaplastic thyroid cancer, rhabdomyosarcoma, breast cancer, colorectal cancer, esophageal cancer (esophagus cancer), renal cell carcinoma, mantle cell lymphoma, chronic lymphocytic leukemia/small lymphocytic leukemia, chronic lymphocytic leukemia/small lymphocytic leukemia with 17p deletion, macroglobulinemia, margin zone lymphoma, chronic graft-versus-host disease, FAK overexpression solid tumor, systemic lupus erythematosus (SLE) and rheumatoid arthritis (RA).

In a preferred embodiment, the disease is selected from the group consisting of esophageal cancer, systemic lupus erythematosus (SLE) and rheumatoid arthritis (RA).

In a preferred embodiment, the disease is selected from the group consisting of esophageal cancer, and the esophageal cancer is preferably classified into an EGFR expression type, HER2 expression type, or myc-amplified esophageal squamous cell carcinoma.

In a preferred embodiment, the disease is selected from EGFR expression type in esophageal squamous cell carcinoma.

In a preferred embodiment, the disease is selected from HER2 expression in type esophageal squamous cell carcinoma.

In a preferred embodiment, the disease is selected from myc-amplified esophageal squamous cell carcinoma.

In some embodiments, the FAK inhibitor or a pharmaceutically acceptable salt or solvate thereof is administrated in an amount of about 0.0025 to 5000 mg/day, such as about 0.005, 0.05, 0.5, 5, 10, 20, 30, 40, 50, 100, 120, 150, 200, 250, 300, 350, 400, 450, 480, 500, 550, 600, 650, 700, 750, 800, 850, 900, 950, 1000, 1500, 2000, 2500, 3000, 3500, 4000, 4500, or 5000 mg/day.

In some embodiments, the FAK inhibitor or a pharmaceutically acceptable salt or solvate thereof is administrated in an amount of about 1 ng/kg to about 200 mg/kg, about 1 μg/kg to about 100 mg/kg or about 1 mg/kg to about 50 mg/kg per unit dose; for example, administrated in an amount of at about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 4 mg/kg, about 5 mg/kg, about 8 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg per unit dose, and one or more (e.g. 1, 2, 3, 4, 5, 6, 7, 8, 9, or 10) unit doses are administrated per day.

In some embodiments, the BTK inhibitor or a pharmaceutically acceptable salt or solvate thereof is administrated in an amount of about 0.0025 to 5000 mg/day, e.g., administrated in an amount of 0.005, 0.05, 0.5, 5, 10, 20, 30, 40, 50, 100, 120, 150, 200, 250, 300, 350, 400, 450, 480, 500, 550, 600, 650, 700, 750, 800, 850, 900, 950, 1000, 1500, 2000, 2500, 3000, 3500, 4000, 4500, or 5000 mg/day.

In some embodiments, the BTK inhibitor or a pharmaceutically acceptable salt or solvate thereof is administrated in an amount of about 1 ng/kg to about 200 mg/kg, about 1 μg/kg to about 100 mg/kg, or about 1 mg/kg to about 50 mg/kg per unit dose; for example, administrated in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 1.6 mg/kg, about 2 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg per unit dose, and one or more (e.g., 1, 2, 3, 4, 5, 6, 7, 8, 9, or 10) unit doses are administrated per day.

In some embodiments, the FAK inhibitor, or a pharmaceutically acceptable salt or solvate thereof, is administrated in an amount of about 0.0025 to 1500 mg/day. Preferably, the daily dose of the FAK inhibitor is 1 mg, 5 mg, 10 mg, 20 mg, 30 mg, 40 mg, 50 mg, 60 mg, 70 mg, 80 mg, 90 mg, 100 mg, 150 mg, 200 mg, 244 mg, 250 mg, 300 mg, 350 mg, 400 mg, 450 mg, 460 mg, 470 mg, 480 mg, 488 mg, 490 mg, 500 mg, 550 mg, 600 mg, 650 mg, 700 mg, 750 mg, 800 mg, 850 mg, 900 mg, 950 mg, 1000 mg, and a range between the above amounts, for example, 1 mg to 1000 mg, 30 mg to 900 mg, 30 mg to 800 mg, 30 mg to 900 mg, 30 mg to 800 mg, 30 mg to 700 mg, 30 mg to 600 mg, 30 mg to 500 mg, 30 mg to 490 mg, 30 mg to 487 mg, etc. The BTK inhibitor, or a pharmaceutically acceptable salt or solvate thereof, is administrated in an amount of about 0.0025 to 1000 mg/day. Preferably, the daily dose of the BTK inhibitor is 10 mg, 20 mg, 30 mg, 40 mg, 50 mg, 60 mg, 70 mg, 73 mg, 80 mg, 90 mg, 97.6 mg, 100 mg, 122 mg, 150 mg, 200 mg, 250 mg, 300 mg, 350 mg, 400 mg, 450 mg, 460 mg, 470 mg, 480 mg, 488 mg, 490 mg, 500 mg, 550 mg, 600 mg, 650 mg, 700 mg, 750 mg, 800 mg, 850 mg, 900 mg, 950 mg, 1000 mg, and a range between the above amounts, for example, 10 mg to 1000 mg, 20 mg to 950 mg, 30 mg to 900 mg, 50 mg to 650 mg, 60 mg to 600 mg, 70 mg to 450 mg, 73 mg to 400 mg, 73 mg to 550 mg, 73 mg to 522 mg, 97.6 mg to 600 mg, 97.6 mg to 600 mg, 97.6 mg to 700 mg, 97.6 mg to 800 mg, 97.6 mg to 950 mg, 122 mg to 500 mg, 122 mg to 600 mg, 122 mg to 700 mg, 122 mg to 800 mg, 97.6 mg to 900 mg, 73 mg to 1000 mg, etc.

In some embodiments, the FAK inhibitor and/or the BTK inhibitor are administrated together, simultaneously, sequentially, or alternately.

In some embodiments, the time interval between sequential administration of the FAK inhibitor and the BTK inhibitor may be about 1 minute, about 5 minutes, about 10 minutes, about 15 minutes, about 30 minutes, about 45 minutes, about 1 hour, about 2 hours, about 4 hours, about 6 hours, about 12 hours, about 24 hours, about 48 hours, about 72 hours, about 96 hours, about 1 week, about 2 weeks, about 3 weeks, about 4 weeks, about 5 week, about 6 weeks, about 8 weeks, or about 12 weeks.

In some embodiments, the pharmaceutical composition of the present invention in the form of a pharmaceutical composition (preferably, each in the form of a separate dosage unit) containing the FAK inhibitor and the BTK inhibitor may be administrated for, including but not limited to: 1 time, 2 times, 3 times, 4 times, 5 times, or 6 times, as required.

In some embodiments, the pharmaceutical composition of the present invention in the form of a pharmaceutical composition (preferably, in the form of a dosage unit) containing the FAK inhibitor and the BTK inhibitor may be administrated for, including but not limited to: 1 time, 2 times, 3 times, 4 times, 5 times, or 6 times, as required.

In some embodiments, the FAK inhibitor and/or the BTK inhibitor are administered continuously for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, or at least 50 days.

In some embodiments, the FAK inhibitor and/or the BTK inhibitor are administered for one or more (e.g., 1, 2, 3, 4, 5, 6, 7, 8, 9, or 10) treatment courses, wherein each treatment course lasts for at least 1 day, 2 days, 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, or at least 50 days; and the time interval between every two treatment courses is 0, 1, 2, 3, 4, 5, 6, 7, 8, 9, 10 days, 2 weeks, 3 weeks or 4 weeks.

In some embodiments, the FAK inhibitor and/or the BTK inhibitor are administered by the same route (e.g., oral administration) or different routes (e.g., oral and parenteral (e.g., injection) administration), respectively.

In some embodiments, the pharmaceutical composition can be administered via the following manners: oral, buccal, inhalation spray, sublingual, rectal, transdermal, vaginal mucosa, transmucosal, topical, nasal or enteral administration; injection such as intramuscular, subcutaneous, intramedullary injection, and intrathecal administration, direct administration in brain, in situ administration, subcutaneous, intraperitoneal, intravenous, intra-articular synovial, intrathoracic, intrahepatic, intralesional, intracranial, intraperitoneal, nasal administration, or intraocular injection, or other drug delivery manners.

Another aspect of the present invention provides a use of a FAK inhibitor and a BTK inhibitor in the manufacture of a pharmaceutical composition and/or kit for treating a disease, wherein the disease is selected from the group consisting of cancer, chronic autoimmune disorder, inflammatory disease, or proliferative disease.

In some embodiments, the disease is selected from the group consisting of anaplastic large cell lymphoma, non-small cell lung cancer, diffuse large B cell lymphoma, inflammatory myofibroblastoma, anaplastic thyroid cancer, rhabdomyosarcoma, breast cancer, colorectal cancer, esophageal cancer (esophagus cancer), renal cell carcinoma, mantle cell lymphoma, chronic lymphocytic leukemia/small lymphocytic leukemia, chronic lymphocytic leukemia/small lymphocytic leukemia with 17p deletion, macroglobulinemia, margin zone lymphoma, chronic graft-versus-host disease, FAK overexpression solid tumor, systemic lupus erythematosus (SLE) and rheumatoid arthritis (RA).

In a preferred embodiment, the disease is selected from the group consisting of esophageal cancer, systemic lupus erythematosus (SLE) and rheumatoid arthritis (RA).

In a preferred embodiment, the disease is selected from esophageal cancer, and the esophageal cancer is classified into an EGFR expression type, HER2 expression type, or myc-amplified esophageal squamous cell carcinoma.

### Specific Models for Carrying Out the Invention

The present invention is further described below through specific examples and comparative examples. However, it should be understood that these examples and comparative examples are merely used to explain the invention in more details, and should not be understood as limiting the present invention in any ways.

The Compound 5 of the present invention could be prepared according to Example 3 disclosed in WO 2018/044767.

### EXAMPLE 1

### Preparation of 5-chloro-N²-(2-isopropoxy-5-methyl-4-(1-(tetrahydro-2H-pyran-4-yl)-1,2,3,6-tetrahydropyridin-4-yl)phenyl)-N⁴-(2-(isopropylsulfonyl)phenyl)pyrimidine-2,4-diamine (Compound 5)

### Step A: Synthesis of 4-(5-fluoro-2-methyl-4-nitrophenyl)-3,6-dihydropyridine-1(2H)-carboxylic acid tert-butyl ester

4-(4,4,5,5-tetramethyl-1,3,2-dioxaborocyclopentan-2-yl)-3,6-dihydropyridine-1(2H)-carboxylic acidtert-butyl ester (620 mg, 2 mmol), Pd(dppf)Cl₂ (58 mg, 0.08 mmol) and K₂CO₃ (828 mg, 6 mmol) were added to a solution of 1-bromo-5-fluoro-2-methyl-4-nitrobenzene (470 mg, 2 mmol) in DME-H₂O (22 mL, 10:1 mixture). The mixture was stirred at 80° C. for 12 hours under nitrogen gas. The reaction mixture was cooled to room temperature and the product was extracted with ethyl acetate. The solvent was removed under reduced pressure, and the residue was purified by silica gel chromatography with hexane/ethyl acetate (9/1, v/v) to afford the title compound of Step A (640 mg, 95% yield) as a light yellow oil.

¹H NMR (400 MHz, CDCl₃) δ ppm 7.89 (d, J=7.5 Hz, 1H), 7.02 (d, J=11.5 Hz, 1H), 5.68 (s, 1H), 4.10-4.07 (m, 2H), 3.65 (t, J=5.6 Hz, 2H), 2.39-2.32 (m, 2H), 2.33 (s, 3H), 1.52 (s, 9H).

### Step B: Synthesis of 4-(5-isopropoxy-2-methyl-4-nitrophenyl)-3,6-dihydropyridine-1(2H)-carboxylic acid tert-butyl ester

To 20 mL of solution of 4-(5-fluoro-2-methyl-4-nitrophenyl)-3,6-dihydropyridine-1(2H)-carboxylic acid tert-butyl ester (640 mg, 1.9 mmol) in 2-propanol was added Cs₂CO₃ (1.862 g, 5.7 mmol). The mixture was stirred at 60° C. overnight and cooled to room temperature, then most of 2-propanol was distilled off under reduced pressure. Water and ethyl acetate were added for extraction. The organic layers were combined, dried over anhydrous Na₂SO₄, concentrated, and the crude product was purified by silica gel chromatography using hexane/ethyl acetate (8/2, v/v) to afford the title compound of Step B (650 mg, 91%) as a yellow oil.

¹H NMR (400 MHz, CDCl₃) δ 7.63 (s, 1H), 6.79 (s, 1H), 5.62 (s, 1H), 4.65-4.62 (m, 1H), 4.10-4.07 (m, 2H), 3.64 (t, J=5.6 Hz, 2H), 2.36-2.34 (m, 2H), 2.25 (s, 3H), 1.52 (s, 9H), 1.39 (d, J=6.1 Hz, 6H).

### Step C: Synthesis of 4-(5-isopropxy-2-methyl-4-nitrophenyl)-1-(tetrahydro-2H-pyran-4-yl)-1,2,3,6-tetrahydropyridine

To a solution of 4-(5-isopropoxy-2-methyl-4-nitrophenyl)-5,6-dihydropyridine-1(2H)-carboxylic acid tert-butyl ester (217 mg, 0.576 mmol) in dichloromethane (5 mL) was addedtrifluoroacetic acid (1 mL), and the reaction mixture was stirred at room temperature for 6 hours. The dichloromethane and trifluoroacetic acid were removed under vacuum, and 100 mL of dichloromethane was added, and washed with a saturated NaHCO₃ solution. The aqueous layer was extracted twice with dichloromethane (100 mL each). The organic layers were combined, washed with brine, dried over Na₂SO₄ and evaporated. The residue was dissolved in dichloromethane (10 mL) and tetrahydro-4H-pyran-4-one (173 mg, 1.728 mmol), and then sodium triacetoxyborohydride (244 mg, 1.152 mmol) and acetic acid (69 mg, 1.152 mmol) were added. The reaction mixture was stirred at room temperature overnight. The reaction was quenched by adding water (80 mL), and extraction was performed with dichloromethane (3×100 mL). The organic layers were combined, washed with brine, dried over Na₂SO₄, concentrated and purified by silica gel column chromatography with ethyl acetate/methanol (9/1, v/v) to afford the title compound of Step C (170 mg, 82%, two Step), which was a yellow oil.

¹H NMR (400 MHz, CDCl₃) δ 7.63 (s, 1H), 6.83 (s, 1H), 5.62-5.59 (m, 1H), 4.58-4.56 (m, 1H), 4.11-4.01 (m, 2H), 3.43-3.28 (m, 4H), 2.78 (t, J=5.6 Hz, 2H), 2.60-2.56 (m, 1H), 2.40-2.36 (m, 2H), 2.23 (s, 3H), 1.86-1.82 (m, 2H), 1.69-1.65 (m, 2H), 1.35 (d, J=6.1 Hz, 6H).

### Step D: Synthesis of 2-isopropoxy-5-methyl-4-(1-(tetrahydro-2H-pyran-4-yl)-1,2,3,6-tetrahydropyridin-4-yl)aniline

To 30 mL of solution of 4-(5-isopropoxy-2-methyl-4-nitrophenyl)-1-(tetrahydro-2H-pyran-4-yl)-1,2,3,6-tetrahydropyridine (2.4 g, 6.66 mmol) in ethanol was added 4 mL of 10% HCl, and then added iron powder (2.23 g, 40 mmol). The mixture was stirred at 60° C. for 3 hours. The reaction mixture was cooled to room temperature and the iron powder was filtered off. The ethanol was removed under reduced pressure to afford the title compound of Step D as a pale yellow oil (2.0 g, 91% yield). MS m/z=331 [M+H].

### Step E: Synthesis of 5-chloro-N²-(2-isopropoxy-5-methyl-4-(1-(tetrahydro-2H-pyran-4-yl)-1,2,3,6-tetrahydropyridin-4-yl)phenyl)-N⁴-(2-(isopropylsulfonyl)phenyl)pyrimidine-2,4-diamine

2-Isopropoxy-5-methyl-4-(1-(tetrahydro-2H-pyran-4-yl)-1,2,3,6-tetrahydropyridin-4-yl)aniline (330 mg, 1 mmol), 2,5-dichloro-N-(2-(isopropylsulfonyl)phenyl)pyrimidin-4-amine (345 mg, 1 mmol), Xantphos (58 mg, 0.1 mmol), Pd(OAc)₂ (11 mg, 0.05 mmol) and Cs₂CO₃ (975 mg, 3 mmol) were dissolved in anhydrous THF (20 mL). N₂ was bubbled through the reaction mixture for 5 minutes, then the reaction vessel was sealed and heated to 150 ° C. under microwave radiation for 30 minutes. The mixture was filtered, and the filtrate was concentrated under reduced pressure. After concentration, the crude product was purified by preparative HPLC (gradient was 10% to 60% acetonitrile in water) to afford the title compound of Step E (125 mg, 20% yield).

¹H NMR (400 MHz, DMSO-d6) δ9.46 (s, 1H), 8.46 (d, J=8.3 Hz, 1H), 8.27 (s, 1H), 8.06 (s, 1H), 7.85 (dd, J=8.3, 1.5 Hz, 1H), 7.66 (t, J=8.3 Hz, 1H), 7.59 (s, 1H), 7.37 (t, J=7.6 Hz, 1H), 6.73 (s, 1H), 5.57-5.50 (m, 1H), 4.58-4.54 (m, 1H), 3.96-3.87 (m, 2H), 3.47-3.43 (m, 1H), 3.31 (t, J=11.1 Hz, 2H), 3.17 (d, J=3.1 Hz, 2H), 2.70 (t, J=5.5 Hz, 2H), 2.29 (t, J=4.5 Hz, 2H), 2.07 (s, 3H), 1.78-1.74 (m, 2H), 1.49-1.45 (m, 2H), 1.23 (d, J=6.0 Hz, 6H), 1.16 (d, J=6.8 Hz, 6H).

The obtained Compound 5 was prepared as a mesylate salt thereof for the use in the following examples. That is, the Compound 5 mentioned in Examples 2 to 6 was used as the mesylate salt of Compound 5.

### EXAMPLE 2

### General Experimental Method Used in the Present Invention

### (1) Determination of Cell Viability and Growth Inhibition by CCK-8 Assay and Colony Formation Assay

Cell viability was measured using Cell Counting Kit-8 (Dojindo, Japan) according to the manufacturer's instructions. In short, cells were inoculated in a 96-well plate at 3000 to 4000 cells/well in the presence of a single drug or a combination of drugs for 72 hours. After 72 hours, CCK-8 reagent (10 μL/well) was added and incubated at 37° C. for 1-2 hours, and absorbance readings at 450 nm were obtained. The IC₅₀ values were calculated by using GraphPad Prism version 6.0.0 (GraphPad Software, San Diego, Calif. USA) for Windows.

Growth inhibition was detected by colony formation assay. ESCC cells were inoculated in 6-well plates at about 500 cells/well, and treated with different inhibitors or DMSO, and then replaced with fresh medium containing with different inhibitors or DMSO every 3 to 4 days. The cells were stained with crystal violet (V5265, Sigma, St Louis, Mo., USA) on day 12. After washing twice with PBS, the cells were fixed with 1% paraformaldehyde and incubated for 15 minutes at room temperature. The cells were then washed twice with PBS and stained with 0.5% crystal violet for 15 minutes at room temperature.

### (2) Detection of Migration Ability by Cell Migration (Transwell) Test

ESCC cells were suspended in FBS-free medium containing Compound 5, Ibrutinib, or both (200 μl cell suspension, 1×10⁵ cells/ml). The culture medium was sucked into a chamber of Transwell (PC membrane, pore size 8.0 μm, Corning, N.Y., USA), and then the chamber was placed in a 24-well plate containing 750 μl of 50% FBS medium. After incubation for 24 to 30 hours, the chamber was taken out, the medium in the chamber was sucked out, and the redundant cells in the chamber were wiped off with a cotton swab. The chamber was placed in paraformaldehyde to perform fixation for 15 minutes, and staining was performed with 0.5% crystal violet at room temperature for 15 minutes. Cell migration activity was described as a relative number of the cells that exited the chamber.

### (3) Analysis of PI-Stained Cell Cycle by Flow Cytometry

For cell cycle analysis, ESCC cells were plated in 6-well plates at 4×10⁵ cells per well, and DMSO, Compound 5, Ibrutinib or both agents were added. After 24 hours of treatment, the cells were collected, added to 70% ethanol and fixed at 4° C. overnight, and then stained with propidium iodide solution (KeyGen Biotech, Nanjing, China) according to the instructions of the KeyGen cycle kit. DNA content was analyzed using ACEA NovoCyte™ flow cytometer (ACEA Biosciences Inc. China).

### (4) Detection of Apoptosis by Flow Cytometry

For the detection of apoptosis experiments, ESCC cells were plated in 12-well plates at 1×10⁵ cells per well, and DMSO, Compound 5, Ibrutinib or both agents were added. After 48 hours of treatment, the cells were collected and washed twice with PBS. The cell staining was carried out according to the instructions of Annexin V-FITC/PI apoptosis detection kit of Beijing Sizhengbai Biotechnology Co., Ltd.: the 4× binding buffer provided from the kit was diluted to 1× with distilled water, and to each sample was added 100 μL of 1× binding buffer to resuspend the cells, then added 5 μL of Annexin V-FITC for incubation in the dark for 5 min; 10 μL of Propidiom Iodide and 400 μL of 1× binding buffer were added and mixed. Samples were placed within 1 h in the dark at room temperature for detection, which was performed on the machine of by using ACEA NovoCyte™ flow cytometer (ACEA Biosciences Inc. China).

### (5) Western Blot Analysis for Mechanism Exploration

After being treated with DMSO, Compound 5, Ibrutinib or both agents, the cells were collected after 24 hours of treatment and washed once with pre-cooled PBS. The cells were lysed on ice with 1× cell lysis buffer containing 1% protease inhibitor (PMSF) and 1% phosphokinase inhibitor for 30 minutes, centrifuged at 12000 rpm and 4° C. for 15 minutes, and the supernatant protein lysate was collected. The protein concentration was measured by BCA protein concentration detection kit. The cell protein lysates (20 to 50 μg) were separated using 8-12% SDS-PAGE electrophoresis. The separated proteins were transferred to a PVDF membrane. The PVDF membrane was blocked with 5% BSA buffer at room temperature for 30 minutes to 1 hour, and then incubated overnight on a 4° C. shaker with 1× TBST containing primary antibody. The redundant primary antibody was eluted, and the membrane was washed 3 times with 1× TBST, 5 to 10 minutes for each time. The protein band membrane was incubated with horseradish peroxidase-labeled secondary antibody dilution for 1 hour at room temperature, and the membrane was washed 3 times with 1× TBST, 5 to 10 minutes for each time. The signal generation and detection were performed using an ECL chemiluminescence ultrasensitive colorimetric kit and a chemiluminescence imaging system. The primary antibodies used in this experiment included: EGFR, (CST, Cat.4267S); phosphorylated EGFR (p-EGFR (Tyr1068)), (CST, Cat.3777T); FAK, (CST, Cat.3285S); phosphorylated FAK (p-FAK (Tyr397)), (CST, Cat.8556T); AKT, (CST, Cat.4685S); phosphorylated p-AKT (Ser473), (CST, Cat.9271S); ERK, (CST,Cat.4695S); phosphorylated ERK (p-ERK (Thr202/Tyr204)), (CST, Cat.4370T); phosphorylated MEK (p-MEK (Ser217/221)), (CST, Cat.9121S);BTK, (Immunoway, Cat. YM1294); C-Myc, (CST, Cat. 5605); phosphorylated C-Myc, (CST, Cat. 13748); Cyclin B1 (CST, Cat. 12231S), Cylin D1 (CST, Cat.2978S), GAPDH (ABGENT, Cat.AM1020B). The secondary antibodies used in this experiment included goat-anti-rabbit secondary antibody (Senta, Cat. Sc-2004) and goat-anti-mouse secondary antibody (Senta, Cat. Sc-2005).

### (6) Establishment of Xenograft Tumor Model for ESCC Cell Line KYSE-150 for the Evaluation of Synergistic Antitumor Effect In Vivo

### Experimental Animal

The animals used were BALB/c Nude mice, 4-6 weeks old, female. Animal body weight was 14-16±20% g. The experimental animals were provided by Beijing Weitonglihua Experimental Animal Technology Co., Ltd. (Vital River Laboratories, VRL, license number: SOCK (J) 2016-0011). Animal certificate number: 11400700325794.

### Feeding Conditions

The experimental animals were raised in the SPF laboratory in the Animal Experiment Building in the North Campus of Sun Yat-sen University. The operation and management of the experimental animals strictly followed the guidelines for Institutional Animal Care and Use Committee (IACUC) of Sun Yat-sen University. All animal experiments were performed under the guidance of IACUC of Sun Yat-sen University. Seven mice were raised in each cage. Each cage was carried with an identification card labeled with the research topic, experimental group, species, gender, and experimental number. The animals were labeled with mouse ear tags. Daily temperature range: 20 to 24° C. Daily humidity range: 40% to 70%. Light: 12 hours for alternate day and night. The animals took food and pure water freely.

### In Vivo Experiment

Compound 5 was provided by Jiangsu Ascentage Pharma Group Corp. Ltd. Compound 5 was dissolved in 20% PG (propylene glycol)/80% NaH₂PO₄ buffer, and diluted to a final concentration of 100 mg/ml according to the experimental protocol, and the final solution was a clear solution. Compound 5 was intragastrically administered at a dose of 100 mg/kg and a volume of 200 μl. The preparation for the administration was prepared every 3 days and stored at 4° C. when it has not been in use. The formulation and use of the preparation were carried out under sterile conditions. Ibrutinib (purity 99%) was purchased from Jiangsu Aikang Biomedical R&D Co., Ltd. (Nanjing). Ibrutinib was administered by intraperitoneal injection at a dose of 25 mg/kg and a volume of 200 μl (0.2 mL/rat). Ibrutinib was formulated as a suspension solution with 20% PEG 400 (polyethylene glycol 400)/5% EL/PBS, and the agent was dissolved to form a light milky white liquid at 4° C. under ultrasounds. The preparation for the administration was prepared every 3 days and stored at 4° C. The formulation and use of the preparation were carried out under sterile conditions.

### Establishment of Model

Forty immunodeficiency mice were subcutaneously injected with 5×10⁶ KYSE-150 cells suspended in 100 μl PBS in the right armpit to establish a xenograft tumor model. After about one week of the inoculation, twenty-eight mice developed transplanted tumor. When the tumor reached an appropriate size (50 to 100 mm³), the animals were randomly assigned according to the tumor volume, in which the difference in tumor volume among groups should be less than 10% of the mean value, and at last 7 mice were assigned to each group, and the administration was star ted on the day of group assignment (i.e., d1). The experimental design was shown in Table 1.

TABLE 1
Experimental design

| | Animal | Drug | Administration | | Dosing |
|---|---|---|---|---|---|
| Group | number | administrated | route | Dose | regimen |
| 1 | 7 | Solvent for | p.o.i.p. | — | qd × 5d,withdrawal 2 |
| | | Compound 5, | | | days/week × 2 weeks |
| | | Solvent forIbrutinib | | | |
| 2 | 7 | Compound 5 | p.o. | 100 mg/kg | qd × 5d,withdrawal 2 |
| | | | | | days/week × 2 weeks |
| 3 | 7 | Ibrutinib | i.p. | 25 mg/kg | qd × 5d,withdrawal 2 |
| | | | | | days/week × 2 weeks |
| 4 | 7 | Compound5 | p.o.i.p. | 100 mg/kg | qd × 5d,withdrawal 2 |
| | | + Ibrutinib | | 25 mg/kg | days/week × 2 weeks |

A subcutaneous immunodeficient-mice xenograft tumor model of human tumor was established by cell inoculation method: tumor cells in logarithmic growth phase were collected, counted and resuspended in 1×PBS, and the concentration of cell suspension was adjusted to 5×10⁷/mL. 1 mL syringe (4 gauge needle) was used to inoculate the tumor cells subcutaneously on the right back of immunodeficient mice, 5×10⁶/0.1 mL/mouse. All animal experiments were performed strictly in accordance with the guidelines for Institutional Animal Care and Use Committee (IACUC) of Sun Yat-sen University and Suzhou Yasheng Pharmaceutical Co., Ltd. The calculation of related parameters referred to China NMPA “Technical Guidelines for Nonclinical Research of Cytotoxic Antitumor Drugs”.

Animal body weight and tumor size were measured twice per week during the experiment. The growth of tumor was periodically observed, and when the tumor grew to an average volume of 50 to 100 mm³, the mice were randomly divided into groups for administration according to tumor size and body weight. The status and deaths of the animals were observed every day. The routine monitoring included the effects of tumor growth and drug administration on the normal behaviors of animals, specially included the activity of experimental animals, food and water consumption, weight gain or loss, eyes, hair, and other abnormal conditions. The deaths and clinical symptoms observed during the experiment were recorded in the raw data. The entire operation for administration, measurement of mouse weight and tumor volume was performed in a clean bench. According to the requirements of the experimental protocol, when the long diameter of tumor was greater than 20 mm, the experimental end point was reached and the mice were euthanized. Tumor tissues were collected, weighed and photographed for recording.

Tumor volume (TV) was calculated as: TV=a×b²/2, wherein a and b represented the measured length and width of tumor, respectively. The relative tumor volume (RTV) was calculated as: RTV=Vₜ/V₁, wherein V₁ represented the tumor volume at the time of group assignment and administration, and Vₜ represented the tumor volume on a certain day after administration. The evaluation index of antitumor activity was relative tumor proliferation rate T/C (%), which was calculated by the formula: relative tumor proliferation rate T/C (%)=(T_{RTV}/C_{RTV})×100%, wherein T_{RTV} represented the RTV of the treatment group, and C_{RTV} represented the RTV of the vehicle control group; tumor remission rate (%) was calculated by dividing the number of SD (stable disease), PR (partial regression of tumor), and CR (complete regression of tumor) in tumor-bearing mice after treatment by the total number of mice in this group×100%.

Animal weight change (Change of body weight, %)=(body weight when measured−body weight when group assignment)/body weight when group assignment×100%.

Criteria for therapeutic evaluation: According to China's NMPA “Technical Guidelines for Nonclinical Research on Cytotoxic Antitumor Drugs” (November 2006), effectiveness was determined when T/C (%) value≤40% and statistic analysis showed p<0.05. If mice lost more than 20% of their body weight or the number of drug-related deaths exceeded 20%, the drug dose was considered severely toxic.

Synergistic analysis used the following formula: synergistic factor=((A/C) (B/C))/(AB/C); wherein A=RTV value of single drug A group; B=RTV value of singe drug B group; C=RTV value of vehicle control group; and AB=RTV value of the group of drug A combined with drug B (Clarke R. Issues in experimental design and endpoint analysis in the study of experimental cytotoxic agents in vivo in breast cancer and other models [J]. Breast Cancer Research & Treatment, 1997, 46 (2-3): 255-278). If the synergistic factor was greater than 1, there was a synergistic effect; if the synergistic factor was 1, there was an additive effect; if the synergistic factor was less than 1, there was an antagonistic effect.

### EXAMPLE 3

### Relationship between EGFR Expression and FAK Expression in Esophageal Squamous Cell Carcinoma Cell Lines, and the Effect of Compound 5 Monotherapy and Ibrutinib Monotherapy on Esophageal Squamous Cell Carcinoma Cell Lines

(1) The experimental method was described in item (1) and item (5) of Example 2. The expression difference of FAK gene between normal esophageal tissue and esophageal cancer tissue was determined by analyzing the TCGA database source information in the GEPIA database (http://gepia.cancer-pku.cn/), and the correlation between the FAK gene and the EGFR gene in esophageal cancer was determined by analyzing the TCGA database source information using the Pearson test. The basic protein expression levels for the following proteins in 6 esophageal cancer cell lines (TE-10, TE-1, YES-2, KYSE-520, KYSE-510, KYSE-150) were detected by Western blot method: EGFR, phosphorylated-EGFR (p-EGFR, Tyr1068), FAK, phosphorylated-FAK (p-FAK, Tyr397), BTK, C-Myc, phosphorylated-C-Myc (p-C-Myc), in which f3-tubulin was used as internal reference protein. In the CCK-8 experiment, the proliferation inhibition effect (IC₅₀ values) of Compound 5 and Ibrutinib on 6 esophageal cancer cell lines (TE-10, TE-1, YES-2, KYSE-520, KYSE-510, KYSE-150) was measured.

### (2) Experimental Results

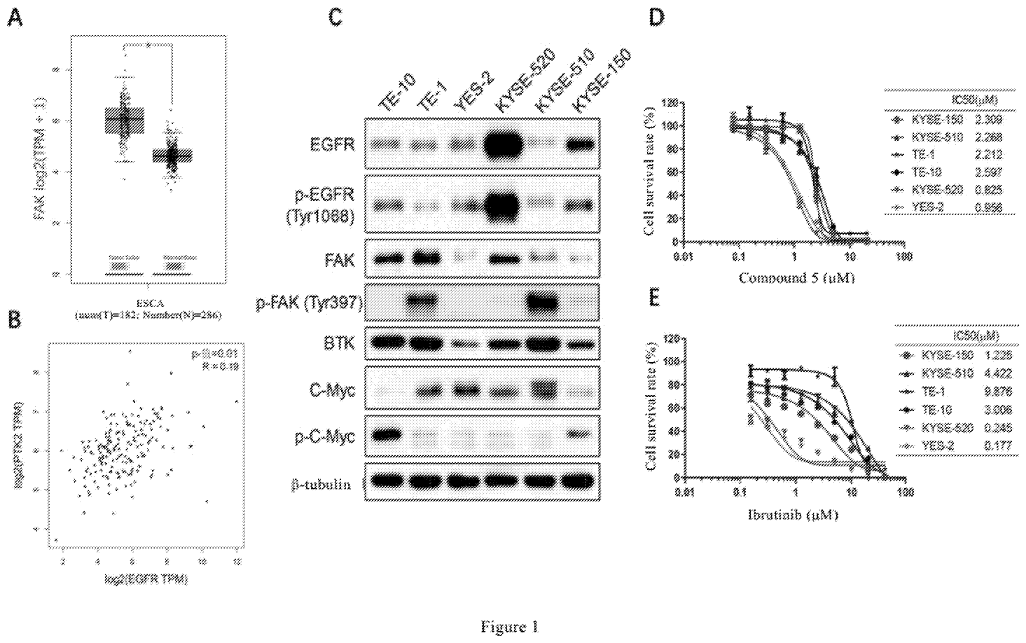

As shown in FIG. 1A, in the data of TCGA, the expression of FAK in esophageal cancer was higher than that in normal esophageal tissue, and FIG. 1B showed that FAK had a positive correlation with the expression of EGFR. FIGS. 1D and E showed that both of Compound 5 and the BTK inhibitor Ibrutinib had good proliferation inhibitory effects on ESCC tumor cells.

Specifically, in FIG. 1C, EGFR was expressed in all of 6 esophageal cancer cell lines, in which KYSE-520 showed the strongest expression, while phosphorylated-EGFR (p-EGFR, Tyr1068) was strongly expressed in KYSE-520, KYSE-150, YES-2 and TE-10. FAK was strongly expressed in TE-10, TE-1 and KYSE-520, phosphorylated-FAK (p-FAK, Tyr397) was strongly expressed in TE-10 and KYSE-510, and BTK was strongly expressed in all of the 6 esophageal cancer cell lines, C-Myc was strongly expressed in TE-1, YES-2, KYSE-520, and KYSE-510, and phosphorylated-C-Myc was expressed in TE-1 and KYSE-510. In FIG. 1D, YES-2 and KYSE-520 cells were more sensitive to Compound 5, with the IC50 values were 0.956 and 0.825 μM, respectively; but for inhibiting the proliferation of TE-10, TE-1, KYSE-510 and KYSE-150, its IC50 values were 2.597, 2.212, 2.268, and 2.309 μM, respectively; in FIG. 1E, the IC50 of ibrutinib single agent for inhibiting proliferation of YES-2 and KYSE-520 was the smallest, 0.177 and 0.245 μM, respectively; and the IC50 values for KYSE-150 and TE-10 cells were 1.225 and 3.006 μM, respectively; but for inhibiting the proliferation of TE-1 and KYSE-510with lower EGFR protein expression, the IC50 were 9.876 and 4.422 μM, respectively.

### (3) Summary

It could be seen that FAK and EGFR were highly expressed in esophageal cancer and had a positive correlation. In vitro experiments showed that Compound 5 had a good proliferation inhibitory effect on ESCC cell lines, while Ibrutinib shown stronger proliferation inhibitory effect in the ESCC cell lines with high EGFR expression.

### EXAMPLE 4

### Effects of Compound 5 and Ibrutinib in Combination on the Proliferation, Cycle Arrest and Apoptosis of Esophageal Squamous Cell Carcinoma Cell Lines In Vitro

(1) The experimental methods were as described in the items (1), (3) and (4) of Example 2. In the CCK-8 experiment, the cell survival rates (%) of the following three esophageal squamous cell carcinoma cell lines (KYSE-150, YES-2, KYSE-520) for Compound 5 and Ibrutinib alone and in combination were determined. In the cell cycle experiment and apoptosis detection experiment, the effects of Compound 5 and Ibrutinib alone and in combination on the changes in cell cycle distribution and the occurrence of apoptosis induction for the following three esophageal squamous cell carcinoma cell lines (KYSE-150, YES-2, KYSE-520) were determined.

### (2) Experimental Results

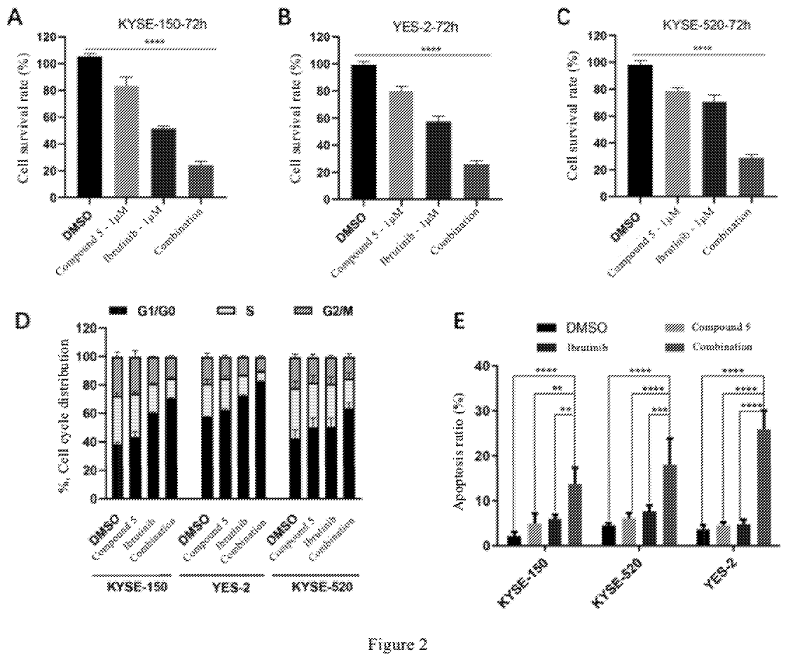

As shown in FIGS. 2A, B, and C, for the 3 esophageal cancer cell lines (KYSE-150, YES-2, and KYSE-520), Compound 5 and Ibrutinib in combination showed an enhanced inhibitory effect on tumor cell proliferation, an increased proportion of cell arrest in GI/GO phase (FIG. 2D), and an increased proportion of apoptosis (FIG. 2E).

Specifically, for the KYSE-150 cells as shown in FIG. 2A, after 1 μM Compound 5 in combination with 1 μM Ibrutinib were administrated to the cells for 72 hours, the inhibitory effect on cell proliferation was further enhanced in comparison with their single drug groups, and the difference was statistically significant (****, p<0.0001; one-way analysis of variance). For the YES-2 cells in FIG. 2B, after 0.5 μM Compound 5 in combination with 0.25 μM Ibrutinib were administrated to the cells for 72 hours, the inhibitory effect on cell proliferation was further enhanced in comparison with their single drug groups, and the difference was statistically significant (****, P<0.0001; one-way analysis of variance). For KYSE-520 cells in FIG. 2C, after 1 μM Compound 5 in combination with 0.5 μM Ibrutinib were administrated to the cells for 72 hours, the inhibitory effect on cell proliferation was further enhanced in comparison with their single drug groups, and the difference was statistically significant (****, P<0.0001; one-way analysis of variance). In the cell cycle experiment as shown in FIG. 2D, after the KYSE-150, YES-2, and KYSE-520 cells were treated at corresponding drug concentrations for 24 hours, the combined drug group showed a significantly upregulated percentage of cell arrest in G1/G0 phase in comparison with the single drug groups. In the detection experiment for cell apoptosis as shown in FIG. 2E, after the KYSE-150, YES-2, and KYSE-520 cells were treated at corresponding drug concentrations for 48 hours, the combined drug group showed an significantly enhanced induction of apoptosis in comparison with the single drug groups, and the difference was statistically significant (**, p<0.01; ***, p<0.001; ****, p<0.0001; one-way analysis of variance).

### (3) Summary

It could be seen that in the in vitro experiments, Compound 5 in combination with Ibrutinib enhanced proliferation inhibitory effect on ESCC cell lines, increased cell cycle arrest, and induced more significant apoptosis.

### EXAMPLE 5

### Effect of Compound 5 and Ibrutinib in Combination on Clone Formation and Cell Migration of Esophageal Squamous Cell Carcinoma Cell Line, and Mechanism Exploration thereof

(1) The experimental method was as described in the items (1), (2), (5) of Example 2. The effects of Compound 5 and Ibrutinib alone and in combination on the cell clone formation ability of the below two esophageal squamous cell carcinoma cell lines (YES-2, KYSE-150) were determined by plate clone formation experiment. The effects of Compound 5 and Ibrutinib alone and in combination on the cell migration ability of the below two esophageal squamous cell carcinoma cell lines (YES-2, KYSE-150) were determined by cell migration Transwell assay. The changes in signaling pathway of related proteins were determined by Western blotting assay.

### (2) Experimental Results

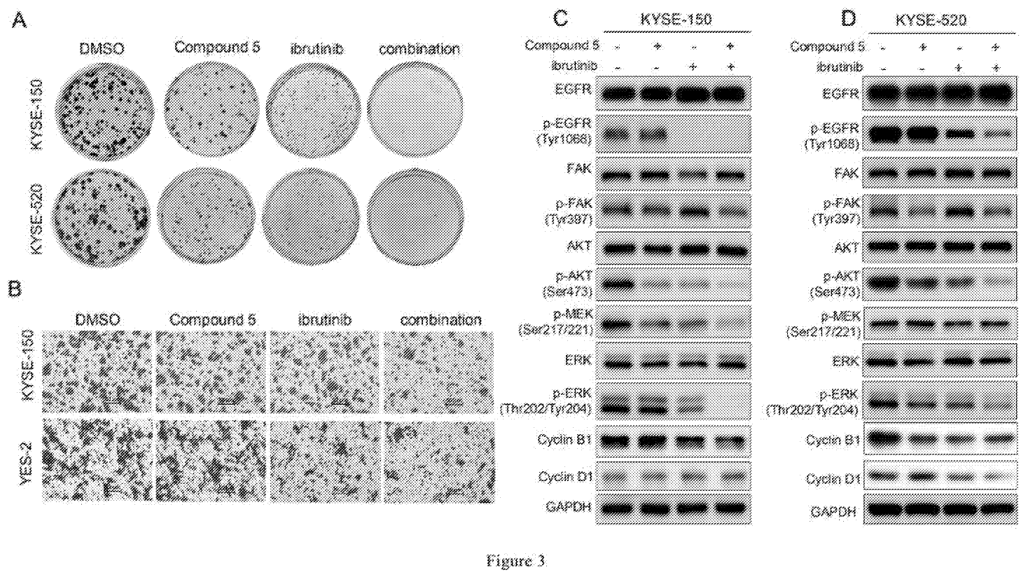

As shown in FIG. 3A, stronger inhibitory effects on cell clone formation were observed in the KYSE-150 cells treated with 1 μM Compound 5 or 1 μM Ibrutinib, and in the YES-2 cells treated with 0.5 μM Compound 5 or 0.25 μM Ibrutinib, but Compound 5 in combination with Ibrutinib produced more significant inhibitory effects on colony formation, while almost no cell clones were formed after continuous culture for 12 days in the combined drug group. As shown in FIG. 3B, cell migration in certain extent was observed in the KYSE-150 cells treated with 1 μM Compound 5 or 1 μM Ibrutinib, and in the YES-2 cells treated with 0.5 μM Compound 5 or 0.25 μM Ibrutinib, but Compound 5 in combination with Ibrutinib produced more significant inhibitory effects on cell migration. As shown in FIGS. 3C and D, by Western blotting assay, the results of the KYES-150 cells treated with 1 μM Compound 5 or 1 μM Ibrutinib and the KYSE-520 cells treated with 1 μM Compound 5 or 0.5 μM Ibrutinib showed that Compound 5 alone could significantly decrease the protein expression levels of phosphorylated-FAK (p-FAK, Tyr397) and phosphorylated-AKT (p-AKT, Ser397), Ibrutinib alone could significantly decrease the protein expression level of phosphorylated EGFR (p-EGFR, Tyr1068), phosphorylated MEK (p-MEK, Ser217/221), phosphorylated ERK (p-ERK, Thr202/Tyr204), Cyclin B1 and Cyclin D1 but Compound 5 and Ibrutinib in combination could more significantly decrease the protein expression level of phosphorylated-AKT (p-AKT, Ser397), MEK (p-MEK, Ser217/221), phosphorylated ERK (p-ERK, Thr202/Tyr204) and Cyclin D1 in comparison with their single drug groups.

### (3) Summary

It could be seen that the combined administration of Compound 5 and Ibrutinib could produce stronger cell clone inhibition effect and cell migration inhibition effect in comparison with their single drug groups. The combined administration of Compound 5 and Ibrutinib could significantly reduce the protein expression level of phosphorylated-AKT, phosphorylated-MEK and phosphorylated-ERK.

### EXAMPLE 6

### Effects of Compound 5, Ibrutinib, and Compound 5 in Combination with Ibrutinib on Mouse Xenograft Tumor Model for Esophageal Squamous Cell Carcinoma KYSE-150 Cell Line from Human Resource

(1) The experimental method was described in the item (6) of Example 2. This experiment evaluated the therapeutic effects of Compound 5 and Ibrutinib alone and in combination in a xenograft model for esophageal squamous cell carcinoma KYSE-150 cell line derived from human. In short, when the average tumor volume reached about 90 mm³, the mice were assigned and administration was started. Compound 5 was administered at a dose of 100 mg/kg, p.o., and Ibrutinib was administered at a dose of 25 mg/kg, i.p., which were all qd administration, after continuous administration for 5 days, withdrawal was performed for 2 days, and then continuous administration was performed again for 5 days. In addition, a group for Compound 5 and Ibrutinib in combination (Ibrutinib 25 mg/kg, i.p., qd+Compound 5 100 mg/kg, p.o., qd) was set.

### (2) Experimental Results

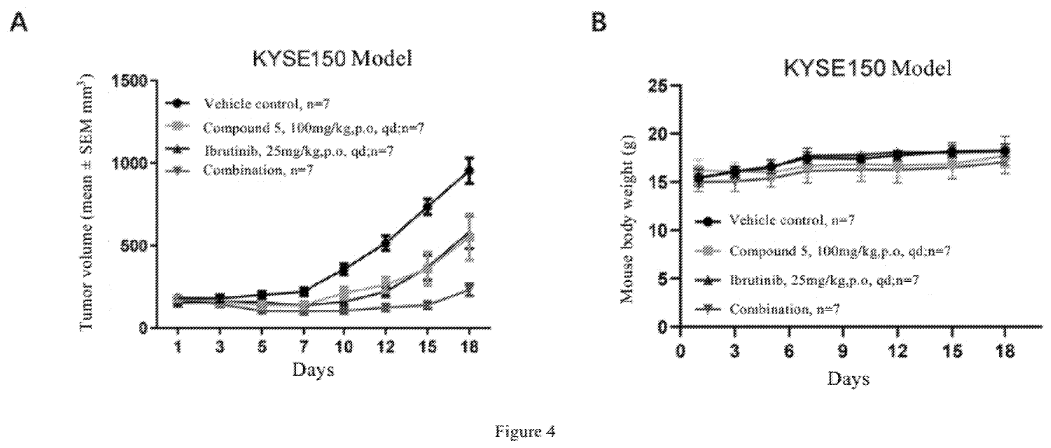

As shown in FIG. 4A, Compound 5 monotherapy and Ibrutinib monotherapy showed inhibitory effects on tumor growth in carcinoma model for esophageal squamous cell KYSE-150from human. The T/C values (%) for the Compound 5 group and the Ibrutinib group after 12 days (last dose) of administration were 49.8% (*p<0.05, compared with the vehicle control) and 48.6% (*p<0.05, compared with the vehicle control), respectively. Both of the Compound 5 monotherapy and Ibrutinib monotherapy showed 1/7 PR and 1/7 SD, and the remission rate was 29%. The combination group of Compound 5 and Ibrutinib significantly enhanced the efficacy, and the T/C value (%) reached 24.9% after 12 days of administration, and had a statistically significant difference (**p<0.01, compared with the vehicle control; *p<0.05, compared with APG-2449 single group; p<0.05, compared with Ibrutinib single group). The combination group showed 5/7 PR and 2/7 SD with a remission rate of 100% (FIG. 4A and Table 2). The synergistic score was 0.97, suggesting no significant synergistic effect. After the drug withdrawal, the advantages of the combination group were more obvious with the extension of time in comparison with Compound 5 monotherapy and Ibrutinib monotherapy. On day 18, the T/C value of the Compound 5 group was 55.8% (p<0.05, compared with the vehicle control), and 1/7 animals maintained PR (remission rate of 14%). The T/C value of the Ibrutinib group was 70.2% (p>0.05, compared with the vehicle control), and no animal maintained PR or SD. The T/C value of the combination of Compound 5 and Ibrutinib remained at 24.9% (*** p<0.001, compared with the vehicle control group; ** p<0.01 compared with the Ibrutinib single group), 2/7 animals maintained PR, 1/7 animals maintained SD, and the remission rate was 43%. The synergistic score>1, suggesting a synergistic effect (FIG. 4A and Table 2). As shown in FIG. 4B, no significant weight change was observed in each of the administration groups, and the animals were in good condition.

TABLE 2
Antitumor effects of Compound 5, Ibrutinib, and Compound 5 in combination with
Ibrutinib in human KYSE-150 mouse xenograft tumor model

| | | | | | | | Tumor | Tumor | | |
|---|---|---|---|---|---|---|---|---|---|---|
| | | | | | | | state on | state on | | |
| | | | | | T/C on | T/C on | day 12 | day 18 | Synergistic | Synergistic |
| | RTV on | | RTV on | | day 12 | day 18 | after | after | score on | score on |
| | day 12 after | | day 18 after | | after | after | admin- | admin- | day 12 | day 18 |
| | administration | | administration | | admin- | admin- | istration | istration | after | after |
| | (mean ± | | (mean ± | | istration | istration | (remission | (remission | admin- | admin- |
| Treatment | standard error) | | standard error) | | (%) | (%) | rate, %^{a}) | rate, %^{a}) | istration | istration |
| Vehicle | 2.9 ± | 0.3 | 5.4 ± | 0.6 | — | — | 0/7 CR | 0/7 CR | — | — |
| control | | | | | | | 0/7 PR | 0/7 PR | | |
| | | | | | | | 0/7 SD | 0/7 SD | | |
| | | | | | | | (0%) | (0%) | | |
| Compound | 1.4 ± | 0.2* | 3.0 ± | 0.7* | 49.8 | 55.8 | 0/7 CR | 0/7 CR | — | — |
| 5 | | | | | | | 1/7 PR | 1/7 PR | | |
| 100 mg/kg | | | | | | | 1/7 SD | 0/7 SD | | |
| | | | | | | | (29%) | (14%) | | |
| Ibrutinib | 1.4 ± | 0.1* | 3.8 ± | 0.5 | 48.6 | 70.2 | 0/7 CR | 0/7 CR | — | — |
| 25 mg/kg | | | | | | | 1/7 PR | 0/7 PR | | |
| | | | | | | | 1/7 SD | 0/7 SD | | |
| | | | | | | | (29%) | (0%) | | |
| Compound | 0.7 ± | 0.1**$# | 1.3 ± | 0.2***# | 24.9 | 24.9 | 0/7 CR | 0/7 CR | 0.97 | 1.58 |
| 5 + | | | | | | | 5/7 PR | 2/7 PR | | |
| Ibrutinib | | | | | | | 2/7 SD | 1/7 SD | | |
| | | | | | | | (100%) | (43%) | | |

*p < 0.05,
**p < 0.01,
***p < 0.001, compared with the vehicle control group;
$ p < 0.05 compared with the Compound 5 group;
# p < 0.05, compared with the Ibrutinib group;
^{a}remission including CR, PR and SD.
Synergistic score >1, suggesting a synergistic effect.

### (3) Summary

In the xenograft tumor model for human esophageal squamous cell carcinoma KYSE-150, the combination of Compound 5 and Ibrutinib had no obvious side effects, and the antitumor effect of the combination was significantly better than that of Compound 5 or Ibrutinib alone. Therefore, the administration of Compound 5 and Ibrutinib in combination could bring clinical benefits to a patient with esophageal squamous cell carcinoma.

1. A pharmaceutical composition, comprising a FAK inhibitor and a BTK inhibitor.

2. The pharmaceutical composition according to claim 1, wherein the FAK inhibitor is a compound of Formula I: 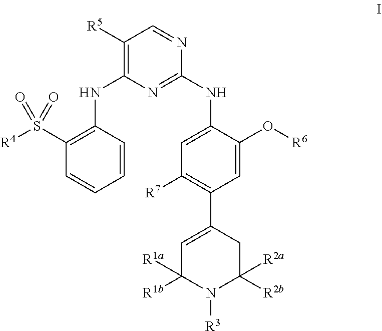
or a pharmaceutically acceptable salt or solvate thereof, wherein:
R^{1a} and R^{1b} are independently selected from the group consisting of hydrogen, C₁₋₆ alkyl and C₃₋₈ cycloalkyl;
R^{2a} and R^{2b} are independently selected from the group consisting of hydrogen, C₁₋₆ alkyl and C₃₋₈ cycloalkyl;
R³ is selected from the group consisting of hydrogen, C₁₋₆ alkyl, C₃₋₆ cycloalkyl, and 4- to 8-membered heterocyclyl;
R⁴ is selected from the group consisting of C₁₋₄ alkyl and C₃₋₆ cycloalkyl;
R⁵ is halogen;
R⁶ is selected from the group consisting of C₁₋₄ alkyl and C₃₋₆ cycloalkyl; and
R⁷ is selected from the group consisting of hydrogen, C₁₋₄ alkyl and C₃₋₆ cycloalkyl;
with proviso that when R^{1a}, R^{1b}, R^{2a} and R^{2b} are each hydrogen, then R³ is selected from the group consisting of C₃₋₆ cycloalkyl and 4- to 8-membered heterocyclyl.

3. The pharmaceutical composition according to claim 2, wherein the FAK inhibitor is a compound of Formula II: 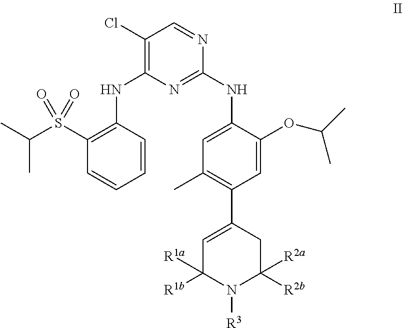
or a pharmaceutically acceptable salt or solvate thereof, wherein:
R^{1a} and R^{1b} are independently selected from the group consisting of hydrogen, C₁₋₄ alkyl and C₃₋₆ cycloalkyl;
R^{2a} and R²b are independently selected from the group consisting of hydrogen, C₁₋₄ alkyl and C₃₋₆ cycloalkyl; and
R³ is selected from the group consisting of hydrogen, C₁₋₄ alkyl, C₃₋₆ cycloalkyl, and 4- to 8-membered heterocyclyl.

4. The pharmaceutical composition according to claim 3, wherein the FAK inhibitor is a compound of Formula III: 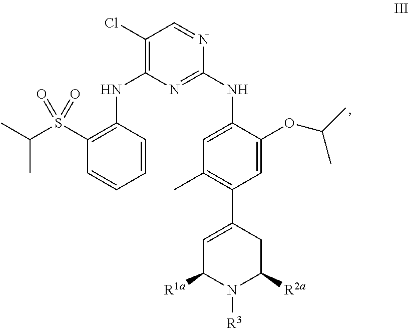
or a pharmaceutically acceptable salt or solvate thereof, wherein:
R^{1a} and R^{2a} are each independently selected from the group consisting of C₁₋₄ alkyl and C₃₋₆ cycloalkyl; and
the compound has an enantiomeric excess of 90% or higher.

5. The pharmaceutical composition according to claim 3, wherein the FAK inhibitor is a compound of Formula IV: 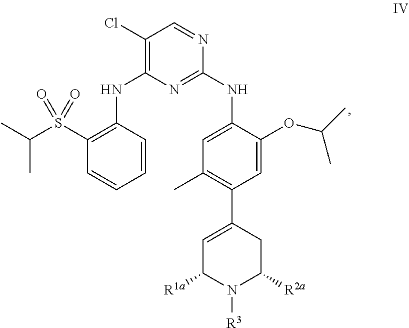
or a pharmaceutically acceptable salt or solvate thereof, wherein:
R^{1a} and R^{2a} are each independently selected from the group consisting of C₁₋₄ alkyl and C₃₋₆ cycloalkyl; and
the compound has an enantiomeric excess of 90% or higher.

6. The pharmaceutical composition according to claim 3, wherein the FAK inhibitor is a compound of Formula V: 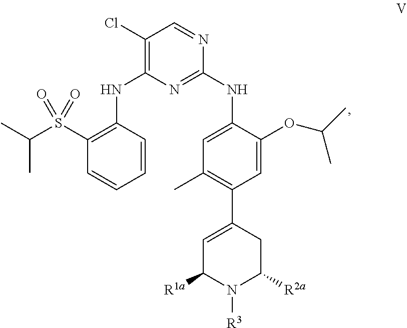
or a pharmaceutically acceptable salt or solvate thereof, wherein:
R^{1a} and R^{2a} are each independently selected from the group consisting of C₁₋₄ alkyl and C₃₋₆ cycloalkyl; and
the compound has an enantiomeric excess of 90% or higher.

7. The pharmaceutical composition according to claim 3, wherein the FAK inhibitor is a compound of Formula VI: 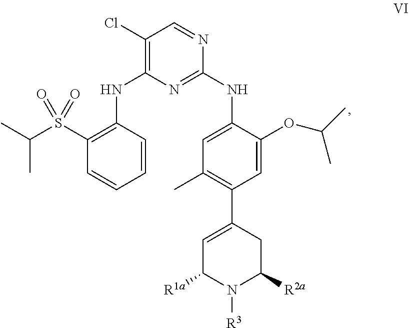
or a pharmaceutically acceptable salt or solvate thereof, wherein:
R^{1a} and R^{2a} are each independently selected from the group consisting of C₁₋₄ alkyl and C₃₋₆ cycloalkyl; and
the compound has an enantiomeric excess of 90% or higher.

8. The pharmaceutical composition according to claim 1, wherein the FAK inhibitor is: 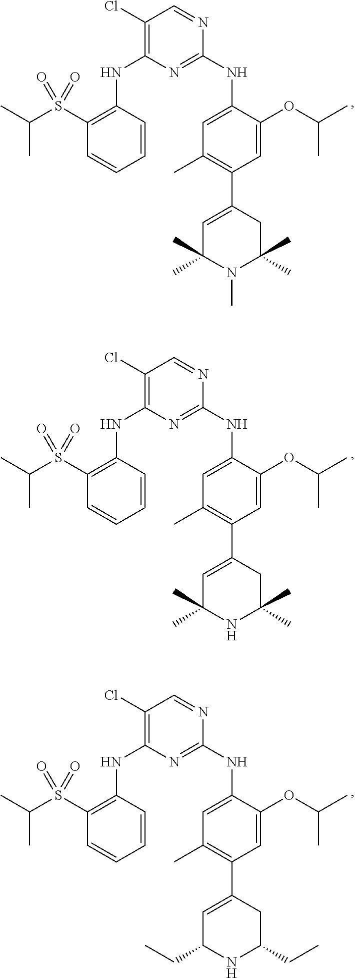 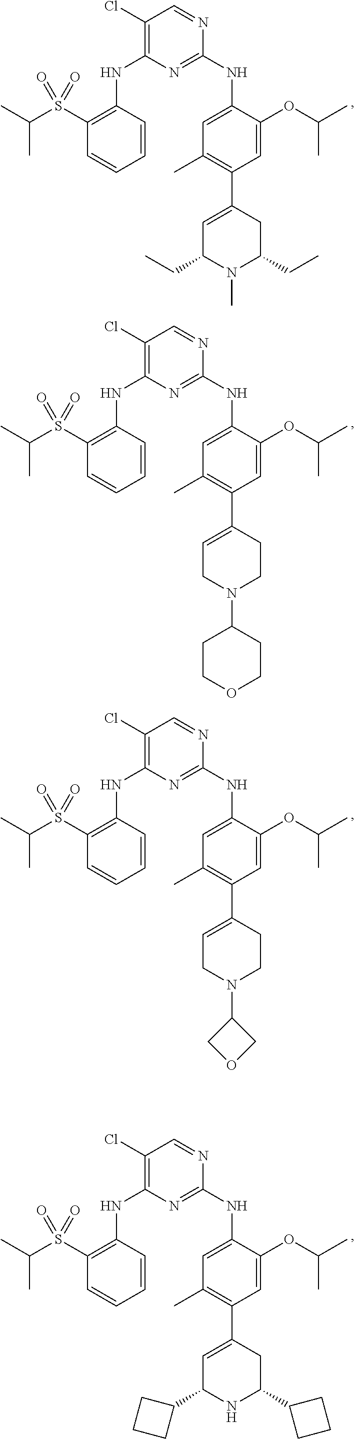 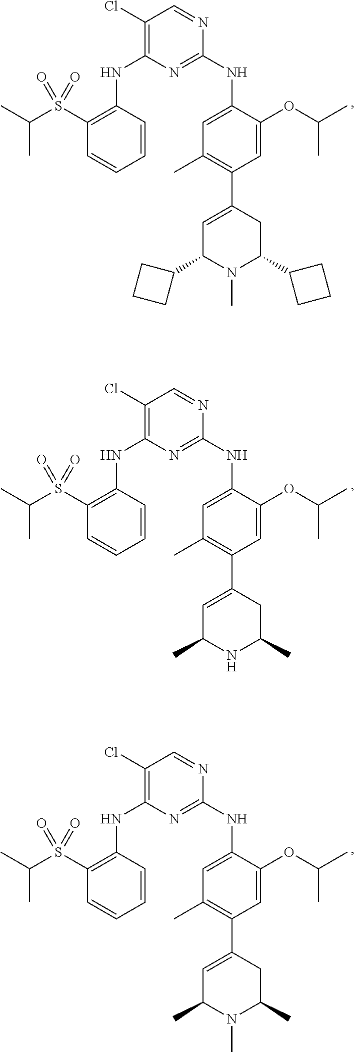 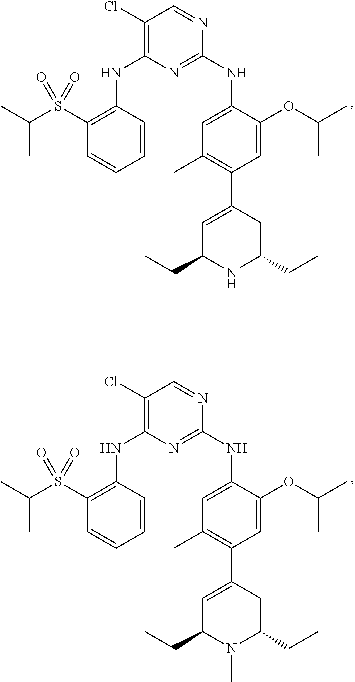 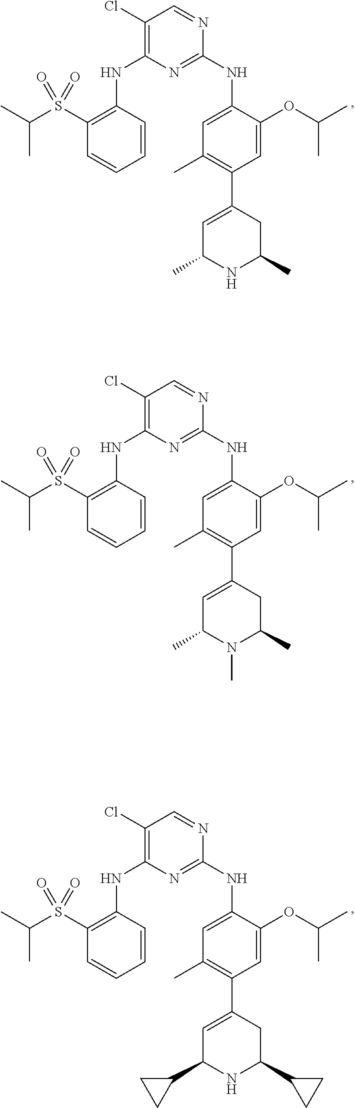 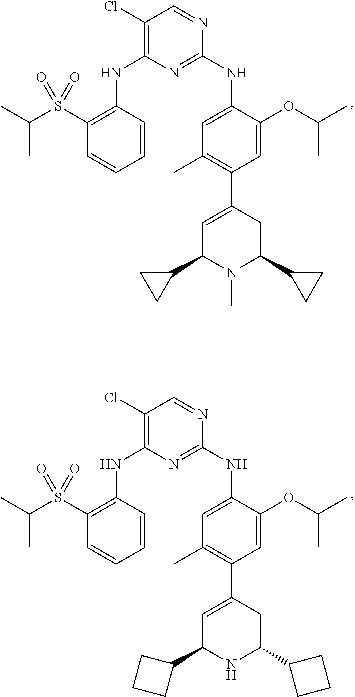 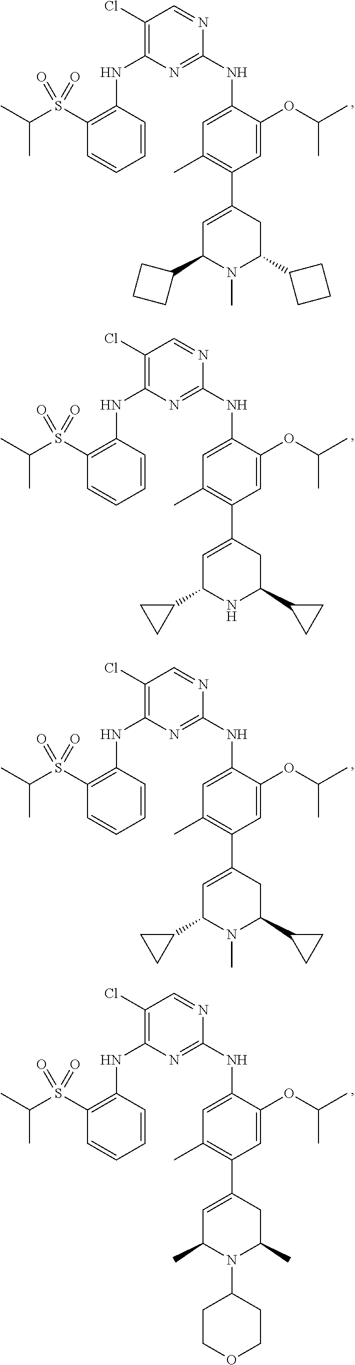 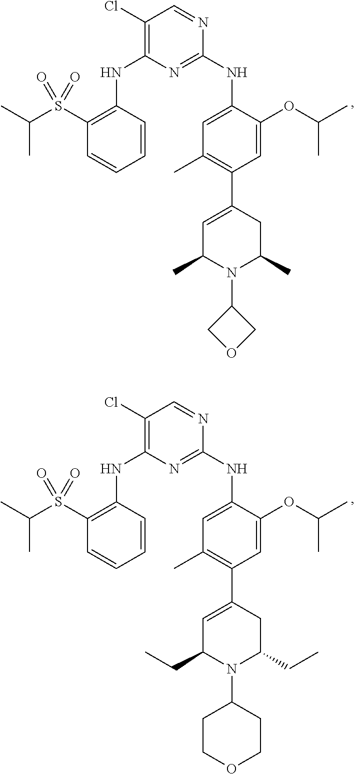 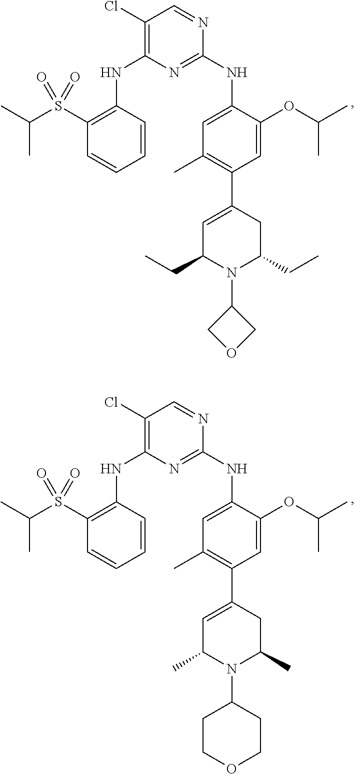 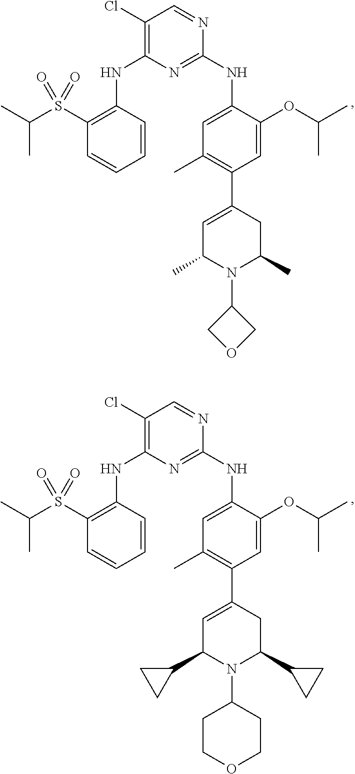 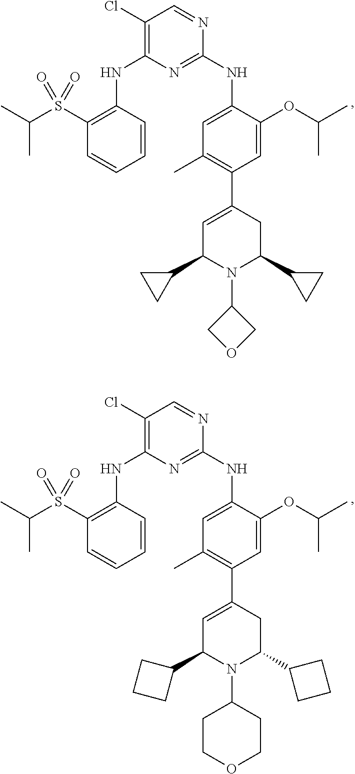 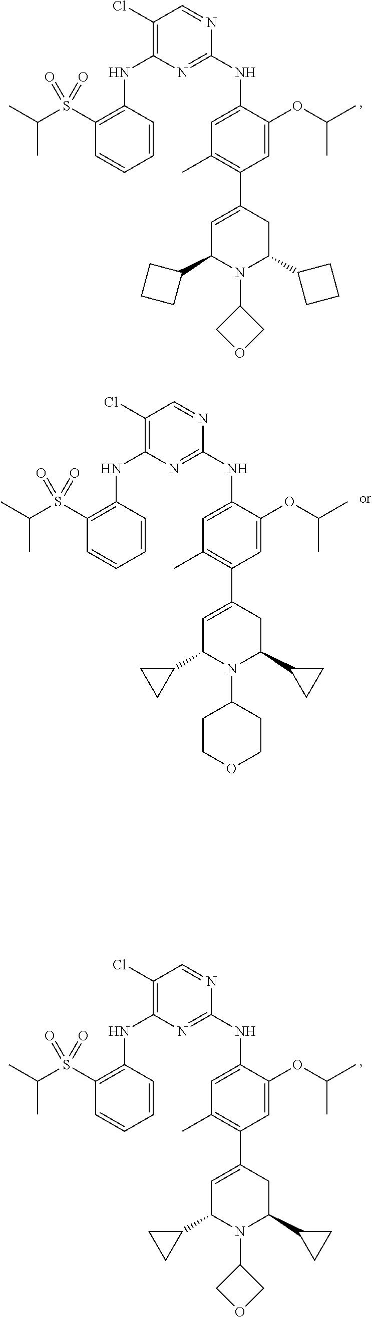
or a pharmaceutically acceptable salt or solvate thereof.

9. The pharmaceutical composition according to claim 1, wherein the FAK inhibitor is 5-chloro-N²-(2-isopropoxy-5-methyl-4-(1-(tetrahydro-2H-pyran-4-yl)-1,2,3,6-tetrahydropyridin-4-yl)phenyl)-N⁴-(2-(isopropylsulfonyl)phenyl)pyrimidine-2,4-diamine, or a pharmaceutically acceptable salt or solvate thereof.

10. The pharmaceutical composition according to claim 1, wherein the BTK inhibitor is selected from the group consisting of: Ibrutinib, ICP-022, Acalabrutinib, BGB3111, ONO/GS-4059, Spebrutinib, CNX-774, Olmutinib, M7583, HM71224, PCI-32765 Racemate, GDC-0853, ONO-4059, Zanubrutinib, RN486, PCI-32765, CGI-1746, QL47, LFM-A13, (±)-Zanubrutinib, SNS-062, BMS-935177, Btk inhibitor 2, Evobrutinib, Ibrutinib-biotin, BMX-IN-1, GDC-0834 and CB1763.

11. The pharmaceutical composition according to claim 1, further comprising a pharmaceutically acceptable carrier, diluent or excipient.

12. (canceled)

13. A kit, comprising a FAK inhibitor and a BTK inhibitor, wherein the FAK inhibitor is a compound of Formula I: 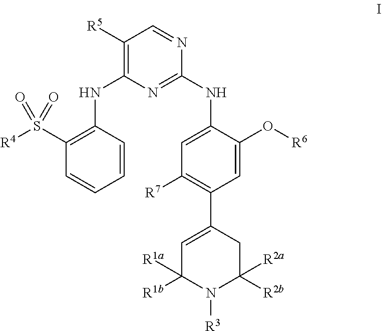
or a pharmaceutically acceptable salt or solvate thereof, wherein:
R^{1a} and R^{1b} are independently selected from the group consisting of hydrogen, C₁₋₆ alkyl and C₃₋₈ cycloalkyl;
R^{2a} and R^{2b} are independently selected from the group consisting of hydrogen, C₁₋₆ alkyl and C₃₋₈ cycloalkyl;
R³ is selected from the group consisting of hydrogen, C₁₋₆ alkyl, C₃₋₆ cycloalkyl, and 4- to 8-membered heterocyclyl;
R⁴ is selected from the group consisting of C₁₋₄ alkyl and C₃₋₆ cycloalkyl;
R⁵ is halogen;
R⁶ is selected from the group consisting of C₁₋₄ alkyl and C₃₋₆ cycloalkyl; and
R⁷ is selected from the group consisting of hydrogen, C₁₋₄ alkyl and C₃₋₆ cycloalkyl;
with proviso that when R^{1a}, R^{1b}, R^{2a} and R^{2b} are each hydrogen, then R³ is selected from the group consisting of C₃₋₆ cycloalkyl and 4- to 8-membered heterocyclyl, and the BTK inhibitor is selected from the group consisting of: Ibrutinib, ICP-022, Acalabrutinib, BGB3111, ONO/GS-4059, Spebrutinib, CNX-774, Olmutinib, M7583, HM71224, PCI-32765 Racemate, GDC-0853, ONO-4059, Zanubrutinib, RN486, PCI-32765, CGI-1746, QL47, LFM-A13, (±)-Zanubrutinib, SNS-062, BMS-935177, Btk inhibitor 2, Evobrutinib, Ibrutinib-biotin, BMX-IN-1, GDC-0834 and CB1763.

14. The kit according to claim 13, wherein the FAK inhibitor is 5-chloro-N²-(2-isopropoxy-5-methyl-4-(1-(tetrahydro-2H-pyran-4-yl)-1,2,3,6-tetrahydropyridin-4-yl)phenyl)-N⁴-(2-(isopropylsulfonyl)phenyl)pyrimidine-2,4-diamine, or a pharmaceutically acceptable salt or solvate thereof, and the BTK inhibitor is Ibrutinib.

15. (canceled)

16. A method for treating a disease, comprising administering a therapeutically effective amount of a FAK inhibitor and a BTK inhibitor to a subject in need thereof, wherein the disease is selected from the group consisting of cancer, chronic autoimmune disease, inflammatory disease and proliferative disease.

17. Currently amended) The method according to claim 16, wherein the FAK inhibitor is
a compound of Formula I: 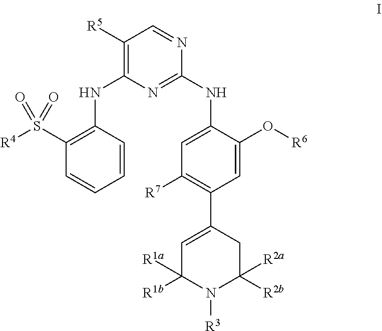
or a pharmaceutically acceptable salt or solvate thereof, wherein:
R^{1a} and R^{1b} are independently selected from the group consisting of hydrogen, C₁₋₆ alkyl and C₃₋₈ cycloalkyl;
R^{2a} and R^{2b} are independently selected from the group consisting of hydrogen, C₁₋₆ alkyl and C₃₋₈ cycloalkyl;
R³ is selected from the group consisting of hydrogen, C₁₋₆ alkyl, C₃₋₆ cycloalkyl, and 4- to 8-membered heterocyclyl;
R⁴ is selected from the group consisting of C₁₋₄ alkyl and C₃₋₆ cycloalkyl;
R⁵ is halogen;
R⁶ is selected from the group consisting of C₁₋₄ alkyl and C₃₋₆ cycloalkyl; and
R⁷ is selected from the group consisting of hydrogen, C₁₋₄ alkyl and C₃₋₆ cycloalkyl;
with proviso that when R^{1a}, R^{1b}, R^{2a} and R^{2b} are each hydrogen, then R³ is selected from the group consisting of C₃₋₆ cycloalkyl and 4- to 8-membered heterocyclyl, and the BTK inhibitor is selected from the group consisting of: Ibrutinib, ICP-022, Acalabrutinib, BGB3111, ONO/GS-4059, Spebrutinib, CNX-774, Olmutinib, M7583, HM71224, PCI-32765 Racemate, GDC-0853, ONO-4059, Zanubrutinib, RN486, PCI-32765, CGI-1746, QL47, LFM-A13, (±)-Zanubrutinib, SNS-062, BMS-935177, Btk inhibitor 2, Evobrutinib, Ibrutinib-biotin, BMX-IN-1, GDC-0834 and CB1763.

18. The method according to claim 17, wherein the FAK inhibitor is 5-chloro-N²-(2-isopropoxy-5-methyl-4-(1-(tetrahydro-2H-pyran-4-yl)-1,2,3,6-tetrahydropyridin-4-yl)phenyl)-N⁴-(2-(isopropylsulfonyl)phenyl)pyrimidine-2,4-diamine, or a pharmaceutically acceptable salt or solvate thereof, and the BTK inhibitor is Ibrutinib.

19. The method according to claim 16, wherein the disease is selected from the group consisting of anaplastic large cell lymphoma, non-small cell lung cancer, diffuse large B cell lymphoma, inflammatory myofibroblastoma, anaplastic thyroid cancer, rhabdomyosarcoma, breast cancer, colorectal cancer, esophageal cancer (esophagus cancer), renal cell carcinoma, mantle cell lymphoma, chronic lymphocytic leukemia/small lymphocytic leukemia, chronic lymphocytic leukemia/small lymphocytic leukemia with 17p deletion, macroglobulinemia, margin zone lymphoma, chronic graft-versus-host disease, FAK overexpression solid tumor, systemic lupus erythematosus (SLE) and rheumatoid arthritis (RA).

20. (canceled)

21. The method according to claim 16, wherein the disease is esophageal cancer, and the esophageal cancer is an EGFR expression type, HER2 expression type, or myc-amplified esophageal squamous cell carcinoma.

22. The method according to claim 16, wherein the FAK inhibitor or a pharmaceutically acceptable salt or solvate thereof is administrated in an amount of about 0.0025 to 5000 mg/day.

23. (canceled)

24. The method according to claim 16, wherein the BTK inhibitor or a pharmaceutically acceptable salt or solvate thereof is administrated in an amount of about 0.0025 to 5000 mg/day.

25-30. (canceled)
